(19) United States
(12) Patent Application Publication
(10) Pub. No.: US 2024/0050392 A1
(43) Pub. Date: Feb. 15, 2024

(54) COMPOSITIONS AND METHODS FOR IMPROVED TREATMENT OF PLATINUM-BASED CHEMOTHERAPEUTIC RESISTANT TUMORS

(71) Applicant: THE REGENTS OF THE UNIVERSITY OF THE COLORADO, A BODY CORPORATE, Denver, CO (US)

(72) Inventors: James C. Costello, Denver, CO (US); Dan Theodorescu, Los Angeles, CA (US); Robert Jones, Denver, CO (US); Tahlita C.M. Zuiverloon, The Hague (NL); Andrew Goodspeed, Denver, CO (US)

(21) Appl. No.: 18/237,705

(22) Filed: Aug. 24, 2023

Related U.S. Application Data
(63) Continuation of application No. PCT/US2022/017991, filed on Feb. 25, 2022.
(60) Provisional application No. 63/153,519, filed on Feb. 25, 2021.

(51) Int. Cl. A61K 31/216; A61K 33/243; A61K 31/282; A61P 35/00; A61K 31/7068; A61K 31/519; A61K 31/475; A61K 31/704

(52) U.S. Cl. CPC A61K 31/216; A61K 33/243; A61K 31/282; A61P 35/00; A61K 31/7068; A61K 31/519; A61K 31/475; A61K 31/704

(57) ABSTRACT

Embodiments of the instant disclosure relate to novel methods and compositions for treating tumors resistant to platinum-based chemotherapy. In certain embodiments, methods of treating tumors herein can include administering an effective amount of at least one M1 aminopeptidase inhibitor in combination with at least one platinum-based chemotherapeutic agent separately or in a combination therapy. In some embodiments, methods of treating tumors disclosed herein can include screening and/or selecting a subject suitable for treatment based on NPEPPS gene or protein expression in the tumor to be treated. In other embodiments, methods of treating tumors can include administering a composition including a combination of at least one M1 aminopeptidase inhibitor and at least one platinum-based chemotherapeutic agent. In certain embodiments, compositions disclosed herein can include at least tosedostat and carboplatin.

### PRIORITY

This application is a U.S. Continuation application of International Application PCT/US2022/017991, filed Feb. 25, 2022, which claims priority to claims priority to U.S. Provisional Application No. 63/153,519 filed Feb. 25, 2021. These applications are incorporated herein by reference in their entirety for all purposes.

### GOVERNMENT FUNDING

This invention was made with government support under Grant No. 5R01CA268055 awarded by the National Institutes of Health. The government has certain rights in this invention.

### FIELD

Embodiments of the instant disclosure relate to novel compositions and methods for treating one or more tumors in a subject having platinum-based chemotherapy resistance or suspected of developing platinum-based chemotherapy resistance.

### BACKGROUND OF THE INVENTION

Platinum-based chemotherapy regimens are frequently a standard of care for cancer patients, particularly those with metastatic disease and/or late stage cancer. A cancerous tumor that responds to a first treatment with drugs containing the metal platinum, such as cisplatin and carboplatin, but then returns within a certain period is considered a platinum-resistant tumor. Patients with platinum-sensitive tumors are often re-treated with platinum; however, a substantial percentage of these patients have a short duration of response after re-treatment and then resistance is developed. For those patients with platinum-resistant disease, positive outcomes are likely to be low. Therefore, knowing whether a cancerous tumor is platinum-resistant can assist in planning further cancer treatments. In addition, treatments are currently sought to maintain tumor sensitivity to standard treatments to tailor a more suitable treatment for a subject to achieve positive results while taking advantage of approved chemotherapeutic agents, for example. As such, there is a need in the art for methods of screening and treating cancer patients to determine more ideal therapeutic treatments to achieve improved outcomes.

### SUMMARY

Embodiments of the instant disclosure relate to novel compositions and methods of use for identifying and/or treating tumors resistant to platinum-based chemotherapy or tumors suspected of developing platinum-based chemotherapy resistance in a subject.

In some embodiments, compositions and methods for treating a subject having a tumor can include administering to the subject a composition including at least one M1 aminopeptidase inhibitor; wherein the subject is undergoing or will undergo an anti-cancer therapy that include, but is not limited to, at least one platinum-based chemotherapeutic. In certain embodiments, compositions and methods disclosed herein can treat a platinum-resistant metastatic solid tumor. In accordance with these embodiments, at least one M1 aminopeptidase inhibitor can be provided to a subject in need thereof, before, during and/or after administering a chemotherapeutic agent to the subject. In certain embodiments, at least one M1 aminopeptidase inhibitor can be provided to a subject having a tumor in advance of platinum-based chemotherapy. In accordance with these embodiments, at least one M1 aminopeptidase inhibitor can be provided to a subject having a tumor immediately before, minutes, half an hour, one hour, hours, days, weeks or months in advance of platinum-based chemotherapy in order to treat the subject.

In some embodiments, compositions and methods disclosed herein can include administering one or more M1 aminopeptidase inhibitors to a subject having a tumor. In certain embodiments, the subject has tumors resistant to platinum-based chemotherapy or tumors suspected of developing platinum-based chemotherapy resistance. In accordance with these embodiments, the one or more M1 aminopeptidase inhibitor can include, but is not limited to, a puromycin-sensitive aminopeptidase (NPEPPS) inhibitor. In other embodiments, a puromycin-sensitive aminopeptidase (NPEPPS) inhibitor can include, but is not limited to, tosedostat, 2-(2,6-Diethylphenyl)-1,2,3,4-tetrahydroisoquinoline-1,3-dione (2: PIQ-22), or similar agent.

In certain embodiments, a tumor to be treated by compositions and methods disclosed herein can be a solid tumor. In some embodiments, solid tumors contemplated herein can include, but are not limited to, a testicular tumor, ovarian tumor, cervical tumor, a kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma, other solid tumor or a combination thereof.

In some embodiments, compositions and methods disclosed herein can include treating a subject with a puromycin-sensitive aminopeptidase (NPEPPS) inhibitor simultaneously, sequentially, or separately from administration of one or more platinum-based chemotherapeutic. In accordance with these embodiments, one or more platinum-based chemotherapeutic agents can include, but is not limited to cisplatin, carboplatin, nedaplatin, satraplatin, picoplatin, phenanthriplatin, triplatin tetranitrate, other platinum-based chemotherapeutic or a combination thereof.

In some embodiments, compositions and methods can include treating a human subject having bladder cancer. In certain embodiments, compositions and methods disclosed herein can include treating the subject with a puromycin-sensitive aminopeptidase (NPEPPS) inhibitor simultaneously, sequentially, or separately from administration of one or more platinum-based chemotherapeutic. In other embodiments, compositions and methods disclosed herein can include treating a human subject having advanced bladder cancer or advance-staged bladder cancer. In certain embodiments, compositions and methods can include treating a human subject having muscle-invasive bladder cancer.

In certain embodiments, NPEPPS expression can be assessed in a subject having cancer (e.g., a solid tumor) contemplated herein according to certain embodiments disclosed herein. In some embodiments, the subject undergoing or scheduled to undergo platinum chemotherapeutic treatment can be assessed for NPEPPS expression in order to assess level of expression compared to a healthy subject not having a tumor or a solid tumor. In accordance with these embodiments, NPEPPS expression in a subject to be treated can be assessed using a tumor biopsy sample from the subject before, during, and/or after a treatment disclosed herein. In some embodiments, NPEPPS expression in the tumor biopsy sample from the subject having a solid tumor can be assessed for level of expression and for assessing whether the level is elevated compared to a control biopsy sample from a healthy subject.

In accordance with these embodiments, changes in NPEPPS expression (e.g., gene expression and/or protein expression/levels) in a subject having or suspected of having a solid tumor can be assessed by comparing a biologic sample (e.g., a tumor biopsy sample, a blood sample, a plasma sample) collected from the subject having or suspected of having a solid tumor to a control biologic sample, wherein the control biologic sample can be from, for example, a matched sample of a subject of the same species (e.g., a human) who are free of the solid tumor. In some examples, a control level can represent the level of NPEPPS expression (e.g., gene expression and/or protein expression/levels) in healthy subjects.

In some embodiments, compositions, and methods for treating a solid tumor as disclosed herein can further include monitoring the subject for the occurrence of one or more adverse effects of treatment or disease progression. In certain embodiments, adverse effects monitored can include, but is not limited to, hepatic impairment, hematologic toxicity, neurologic toxicity, cutaneous toxicity, gastrointestinal toxicity, or a combination thereof. In some embodiments, compositions and methods disclosed herein can include adjusting the dose of the at least one M1 aminopeptidase inhibitor, the dose of the one or more platinum-based chemotherapeutics, or both, when an adverse effect is observed, or tumor regression is not observed.

In certain embodiments, compositions for treating cancer in a subject contemplated herein can include, but is not limited to, one or more M1 aminopeptidase inhibitors and one or more platinum-based chemotherapeutics. In certain embodiments, compositions disclosed herein including one or more M1 aminopeptidase inhibitors can include, but are not limited to, a puromycin-sensitive aminopeptidase (NPEPPS) inhibitor. In some embodiments, compositions contemplated herein, including one or more platinum-based chemotherapeutics (platins) can include, but are not limited to, cisplatin, carboplatin, nedaplatin, satraplatin, picoplatin, phenanthriplatin, triplatin tetranitrate or the like. In other embodiments, compositions for treating cancer or solid tumors for use in methods disclosed herein can include, at least a combination of tosedostat and a platin of carboplatin. In some embodiments, compositions disclosed herein can further include one or more anti-neoplastic agent including, but not limited to, one or more of gemcitabine, methotrexate, vinblastine, and adriamycin and a combination thereof.

In certain embodiments, pharmaceutical compositions are contemplated including, but not limited to, at least one M1 aminopeptidase inhibitor and at least one chemotherapeutic agent and a pharmaceutically acceptable excipient for use in treating a solid tumor. In some embodiments, a pharmaceutical combination composition can include at least one M1 aminopeptidase inhibitor and at least one platinum-based chemotherapeutic agent for manufacturing a composition or medicament. In some embodiments, the pharmaceutical combination composition can be used for treating a solid tumor in a subject in need thereof.

In certain embodiments, kits are provided. In certain embodiments, compositions or combination compositions of the instant disclosure can be included in the kit, and at least one container. In certain embodiments, kits are provided for the practice of any one of the methods disclosed herein. In some embodiments, kits can include one or more M1 aminopeptidase inhibitors, one or more platinum-based chemotherapeutics, or a combination thereof or a pharmaceutical composition thereof.

### BRIEF DESCRIPTION OF THE DRAWINGS

FIGS. 1A-1C illustrate a schematic of treatment of various cancer cell lines with certain treatment regimens and analysis thereof and examples of certain outcomes from pooled CRISPR screens identifying synthetic lethal gene-to-drug relationships in human bladder cancer (BCa) cell lines derived from muscle-invasive bladder cancer (MIBC) patients in accordance with certain embodiments of the present disclosure. FIG. 1A is a schematic depicting the experimental design for molecularly characterizing human bladder cancer cell lines resistant to various agents. FIG. 1B illustrates a heatmap depicting changes in some commonly synthetic lethal genes in response to saline (PBS) or gemcitabine plus cisplatin treatment. FIG. 1C illustrates a graph depicting pathway enrichment across the full synthetic lethal screen results representing genes responsive to treatments in accordance with certain embodiments disclosed herein.

FIGS. 2A-2E illustrate examples of differential gene and protein expression of the 46 common synthetic lethal genes in a cell line as measured by RNAseq in accordance with certain embodiments of the present disclosure. FIG. 2A illustrates a heatmap depicting differential gene expression of common synthetic lethal genes between treatment-resistant cells and treatment-sensitive cells. FIGS. 2B-2D illustrate graphs depicting genes upregulated in treatment-resistant cells as determined by RNAseq (FIG. 2B), mass spectrometry proteomics (FIG. 2C), and synthetic lethality in the CRISPR screen for NPEPPS (FIG. 2D). FIG. 2E illustrates densitometry quantification for NPEPPS protein expression in certain cell lines in accordance with certain embodiments disclosed herein

FIGS. 3A-3H illustrate examples of genetic and pharmacological inhibition of NPEPPS in resistant cells in accordance with certain embodiments of the present disclosure. FIG. 3A illustrates a representative immunoblot for NPEPPS in cells treated with shRNAs targeting NPEPPS. FIGS. 3B-3C illustrate graphs depicting cells with reduced NPEPPS expression treated with increasing doses of cisplatin (FIG. 3B) or gemcitabine (FIG. 3C). FIG. 3D illustrates a graph depicting cells overexpressing NPEPPS treated with increasing doses of cisplatin. FIG. 3E illustrates a graph depicting NPEPPS mRNA upregulatation in response to cisplatin treatment in KU1919-parental and KU1919-GemCis cells. FIGS. 3F-3H illustrate graphs depicting restored sensitivity in tosedostat treated GemCis-resistant KU1919 cells (FIG. 3F), GemCis-resistant T24 cells (FIG. 3G), and GemCis-resistant TCCSUP cells (FIG. 3H) in accordance with certain embodiments disclosed herein.

FIGS. 4A-4F illustrate examples of NPEPPS interacting with volume regulated anion channel (VRAC) subunits to mediate a platin (e.g., cisplatin) response in accordance with certain embodiments of the present disclosure. FIGS. 4A-4B illustrate representative immunoblots of tagged NPEPPS overexpression in KU1919 and T24 parental cell lines (FIG. 4A) and anti-tag-Ab immunoprecipitants from KU1919 and T24 parental cell lines immunoblotted for NPEPPS and LRRC8A (FIG. 4B). FIG. 4C illustrates representative immunoblots of NPEPPS-Ab or LRRC8A-Ab immunoprecipitants from KU1919-GemCis or T24 parental cell lines immunoblotted for NPEPPS and LRRC8A. FIG. 4D illustrates a graph depicting synthetic lethal genes ranked based on log 2 fold change from the synthetic lethal CRISPR screens across all cell lines. LRRC8A-E and common synthetic lethal genes are labeled. FIGS. 4E-4F illustrate graphs depicting the effect of LRRC8A knockout on cell growth in GemCis-resistant cell lines from the CRISPR screen (FIG. 4E) and in GemCis-resistant cell lines with LRRC8D knockout (FIG. 4F) after a combined gemcitabine and cisplatin treatment in accordance with certain embodiments disclosed herein.

FIGS. 5A-5L illustrate examples of genetic and pharmacological inhibition of NPEPPS re-sensitizing in vivo and ex vivo models of bladder cancer to platin (e.g., cisplatin)-based chemotherapy in accordance with certain embodiments of the present disclosure. FIGS. 5A-5B show graphs depicting tumor volume (FIG. 5A) and survival analysis (FIG. 5B) of KU1919-GemCis xenografts contacted with non-targeting shRNA controls (shCtrl1), shRNA targeting NPEPPS (shN39), PBS vehicle control (PBS), or gemcitabine plus cisplatin treatment (GemCis). FIG. 5C illustrates representative immunoblots depicting NPEPPS protein expression in KU1919-GemCis cells injected into mice to establish tumors (left panel) and in tumor samples having an endpoint of a tumor volume >2 cm³ harvested from mice (right panels). FIGS. 5D-5I illustrate representative images and graphs depicting organoid growth days after reseeding at predetermined ratios, Patient 1 (FIG. 5D; cell density quantified in FIG. 5E); Patient 2 (FIG. 5F; cell density quantified in FIG. 5G); and Patient 3 (FIG. 5H; cell density quantified in FIG. 5I). FIG. 5J illustrates a graph depicting NPEPPS gene expression in organoids after several hours of treatment with cisplatin. FIGS. 5K-5L illustrate graphs depicting survival analysis where patients had a record of cisplatin-based chemotherapy treatment (FIG. 5K) or had no record of cisplatin-based chemotherapy treatment (FIG. 5L) in accordance with certain embodiments disclosed herein.

FIG. 6 illustrates a schematic model as an example of NPEPPS-mediated platin (e.g., cisplatin) resistance in accordance with certain embodiments of the present disclosure.

FIGS. 7A-7J illustrates examples of BCa cell line resistance to gemcitabine and/or platin (e.g., cisplatin) concentrations compared to the parental lines in accordance with certain embodiments of the present disclosure. BCa cell lines included: KU1919 cells sensitive to cisplatin (FIG. 7A) and gemcitabine (FIG. 7B); 253J cells sensitive to cisplatin (FIG. 7C) and gemcitabine (FIG. 7D); 5637 cells sensitive to cisplatin (FIG. 7E) and gemcitabine (FIG. 7F); T24 cells sensitive to cisplatin (FIG. 7G) and gemcitabine (FIG. 7H); and TCCSUP cells sensitive to cisplatin (FIG. 7I) and gemcitabine (FIG. 7J) in accordance with certain embodiments disclosed herein.

FIGS. 8A-8B illustrate examples of intracellular cisplatin measured by CyTOF (cytometry by time of flight) in KU1919 cells (FIG. 8A) and in 5637 cells (FIG. 8B) that were treated with PBS vehicle (0 μM) or 10 μM cisplatin in accordance with certain embodiments of the present disclosure.

FIGS. 9A-9B illustrate an example of the loss of LRRC8D gene expression in TCCSUP-Cis and TCCSUP-GemCis cells in accordance with certain embodiments of the present disclosure.

FIGS. 10A-10B illustrate examples of organoid models derived from subjects that failed various cycles of neoadjuvant gemcitabine plus cisplatin chemotherapy in accordance with certain embodiments of the present disclosure. FIG. 10A illustrates a schematic depicting a clinical time course of muscle-invasive bladder cancer patients used for generating the patient-derived organoid lines described herein. FIG. 10B illustrates representative images depicting patient-derived organoids with H&E staining of patient tumors in accordance with certain embodiments disclosed herein.

FIGS. 11A-11F illustrate examples of xenograft tumor growth modeling and validation in mice injected with cells to establish tumors in accordance with certain embodiments of the present disclosure. FIGS. 11A-11D illustrate graphs depicting longitudinal tumor volumes for tumors transduced with shCtrl1 and treated with PBS (FIG. 11A), transduced with shCtrl1 and treated with gemcitabine plus cisplatin chemotherapy (FIG. 11B), tumors transduced with shN39 and treated with PBS (FIG. 11C), and transduced with shN39 and treated with gemcitabine plus cisplatin chemotherapy (FIG. 11D). FIGS. 11E-11F illustrate graphs depicting residuals for the final mixed-effects PBS-treatment model (FIG. 11E) and gemcitabine plus cisplatin chemotherapy model (FIG. 11F) in accordance with certain embodiments disclosed herein.

FIGS. 12A-12K illustrate examples of NPEPPS regulation of VRAC activity and tumor growth in accordance with certain embodiments of the present disclosure. FIG. 12A illustrates a plot depicting metabolites measured from KU1919-GemCis cells with or without NPEPPS knockdown (shN39). FIG. 12B illustrates a graph depicting taurine abundance measured in KU1919-GemCis cells with non-targeting shRNA controls or shRNA targeting NPEPPS (shN39). FIGS. 12C-12E illustrate graphs depicting intracellular cisplatin levels in KU1919 (FIG. 12C), 5637 (FIG. 12D), and T24 (FIG. 12E) cells measured after 4 hours of 10 μM cisplatin treatment using CyTOF. FIGS. 12F-12H illustrate graphs depicting intracellular cisplatin levels in KU1919 (FIG. 12F), 5637 (FIG. 12G), and T24 (FIG. 12H) cells having untargeted knockdown (siCtrl), targeted knockdown of NPEPPS (siNPEPPS), LRRC8A (siLRRC8A), and the combination of NPEPPS and LRRC8A (siNPEPPS+siLRRC8A). FIG. 12I illustrates a representative image depicting an immunoblot of LRRC8A, LRRC8D, and NPEPPS in TCCSUP cells. FIG. 12J illustrates a graph depicting intracellular cisplatin levels in TCCSUP cells. FIG. 12K illustrates a graph depicting intracellular cisplatin was measured in TCCSUP-GemCis cells with overexpression of LRRC8D in accordance with certain embodiments disclosed herein.

FIG. 13 illustrates an example of NPEPPS and LRRC8A knockdown in parental BCa cell lines in accordance with certain embodiments of the present disclosure.

FIGS. 14A-14D illustrate examples of intracellular cisplatin response to NPEPPS and LRRC8A knockdown in GemCis resistant BCa cell lines in accordance with certain embodiments of the present disclosure. FIG. 14A illustrates a representative immunoblot depicting NPEPPS and LRRC8A protein expression in KU1919-GemCis, 5637-GemCis, or T24-GemCis cells with control knockdown (siCtrl) or targeted knockdowns or the combination of targeted knockdown. FIGS. 14B-14D illustrate intracellular cisplatin measurements, performed in two replicates, in KU1919 (FIG. 14B), 5637 (FIG. 14C), and T24 (FIG. 14D) GemCis cells treated with 10 μM cisplatin for 4 hours in accordance with certain embodiments disclosed herein.

FIGS. 15A-15D illustrate examples of intracellular cisplatin response to overexpression of NPEPPS in parental BCa cell lines in accordance with certain embodiments of the present disclosure. FIG. 15A illustrates a representative immunoblot depicting NPEPPS protein expression in parental cells, overexpression control (OE-Ctrl) cells, overexpression (OE-NPEPPS) cells, or KU1919 and T24 GemCis cells. FIGS. 15B-15D illustrate intracellular cisplatin measurements, performed in two replicates, in KU1919 (FIG. 15B), 5637 (FIG. 15C), and T24 (FIG. 15D) cells treated with 10 μM cisplatin in accordance with certain embodiments disclosed herein.

FIGS. 16A-16D illustrate examples of intracellular carboplatin response to NPEPPS and LRRC8A knockdown in parental and GemCis resistant BCa cell lines in accordance with certain embodiments of the present disclosure. FIGS. 16A-16B illustrate intracellular carboplatin measurements or assessments, performed in replicates, in KU1919 parental (FIG. 16A) and T24 parental (FIG. 16B) treated cells. FIGS. 16C-16D illustrate intracellular carboplatin measurements, performed in replicates, in KU1919 GemCis (FIG. 16C) and T24 GemCis (FIG. 16D) treated cells in accordance with certain embodiments disclosed herein.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, pets such as dogs, cats, horses such as performance or work horses, livestock such as cattle, sheep, pigs, rabbits, and goats, chickens, amphibians, reptiles, sea mammals and the like. In some embodiments, the subject is a human subject. In other embodiments, the subject is a human in need of chemotherapeutic intervention for cancer or a solid tumor or other condition typically treated by a platin-based therapy.

As used herein, “treatment,” “therapy” and/or “therapy regimen” can mean clinical intervention made in response to a disease, disorder or health condition manifested by a subject. Treatment can include alleviation or prevention of symptoms, slowing or stopping the progression or worsening of a disease, disorder, or condition and/or the remission of the disease, disorder or condition or shrinking or elimination of an unwanted growth such as a tumor. As used herein, the term “treating” can mean administering a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

As used herein, “prevent” or “prevention” refers to eliminating or delaying the onset of a particular disease, disorder or health condition, or to the reduction of the degree of severity of a particular disease, disorder or health condition, relative to the time and/or degree of onset or severity in the absence of intervention.

The term “effective amount” or “therapeutically effective amount” refers to an amount sufficient to effect beneficial or desirable biological and/or clinical results.

Alleviating a target disease/disorder includes delaying the development or progression of the disease, or reducing disease severity or prolonging survival of a subject. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a condition can mean initial manifestations and/or ensuing progression of the condition. Development of the disease can be detectable and assessed using standard clinical techniques as well known in the art. However, development also refers to progression that can be undetectable. For purpose of this disclosure, development or progression refers to the biological course of the symptoms. “Development” includes, but is not limited to, occurrence, recurrence, and onset. As used herein “onset” or “occurrence” of a target disease or disorder includes initial onset and/or recurrence.

Unless otherwise defined, all technical terms used herein have the same meaning as commonly understood by one of ordinary skill in the art to which this disclosure belongs.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Embodiments of the instant disclosure relate to novel methods and compositions for treating tumors resistant to or suspected of developing resistance to a platinum-based chemotherapy. In some embodiments, a tumor subjected to the methods and compositions disclosed herein can be a solid tumor in a subject having cancer. In some embodiments disclosed herein, compositions and methods are designed to identify issues regarding platinum-based treatments, re-sensitize or sensitize a tumor in a subject to platinum-based chemotherapy to improve outcome and reduce or eliminate patient exposure to platinum-based therapy without significant effect and reduce costs.

In some embodiments, a subject can have a platinum-based chemotherapy resistant tumor or be suspected of developing such a tumor where additional agents contemplated herein can be administered to re-sensitize or sensitize a tumor in the subject to these treatments. In accordance with these embodiments, the tumor can include at least one solid tumor. In certain embodiments, a solid tumor can be an abnormal mass of tissue that is devoid of cysts or liquid regions within the tumor. In other embodiments, solid tumors can be benign (not progressed to a cancer), a malignant or metastatic tumor. In some embodiments, a solid tumor can include a malignant cancer that has metastasized to another region of a subject's body or another organ. In other embodiments, solid tumors contemplated herein can include, but are not limited to, sarcomas, carcinomas, lymphomas, gliomas, other solid tumors, or a combinational thereof. In accordance with these embodiments, tumors resistant to platinum-based chemotherapy can include, but are not limited to, a testicular tumor, ovarian tumor, cervical tumor, a kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma, other solid resistant tumor or a combination thereof. In some embodiments, a targeted tumor contemplated herein can include a solid tumor such as muscle-invasive bladder tumors.

It is known by those of skill in the art that some standards of care for solid tumors include combination therapies but there has been limited success with combination therapies. For example, cisplatin-based combination chemotherapy, (primarily gemcitabine plus cisplatin), is one standard of care in the neoadjuvant (NAC) setting for the management of muscle-invasive bladder cancer (MIBC); however, the adoption of this regimen has been relatively slow due to several factors. These combinations can lead to toxicity of the drugs, significant number of patients that are ineligible for cisplatin-based NAC (about 25%), and a relatively small therapeutic survival benefit of about 5-15% over immediate cystectomy when these combinations are used. Embodiments disclosed herein are designed to avoid such outcomes as there is a need in the art for new combination therapies that are equally or more effective than the current standard of care, and that offer subject a much more tolerable chemotherapeutic regimen with improved outcomes.

Studies have been performed and are currently continuing to identify biomarkers of chemotherapeutically-resistant cancers such as platinum-resistant cancers. Therapeutically actionable targets in platinum-resistant tumors have been hampered for example, by reliance on using simplistic techniques with tumor-derived cell line models. Embodiments of the instant inventions are based, at least in part, on the identification of genes and pathways critical for chemoresistance in cancer as identified through for example, multi-omic molecular profiling, coupled with whole genome CRISPR screening under treatment. Using these methods of advanced profiling, the present disclosure provides the surprising discovery that upregulation of puromycin-sensitive aminopeptidase, NPEPPS, is a novel mechanism of tumors that are chemotherapeutic resistant. In certain embodiments, it was observed that upregulation of NPEPPS is a novel mechanism of tumors that are chemotherapeutic resistant to, for example, gemcitabine plus cisplatin. Certain embodiments disclosed herein use novel methods and compositions for treating tumors resistant to platinum-based chemotherapy by, for example, identifying a subject having tumors with increased expression of NPEPPS and/or administering an effective amount of at least one M1 aminopeptidase inhibitor to reduce expression of NPEPPS in a targeted tumor in combination with at least one platinum-based chemotherapeutic to the subject for treating a solid tumor. In some embodiments, these treatments can be used whether or not the tumor has been identified as a NPEPPS over-expresser.

In some embodiments, compositions of use herein can include one or more M1 aminopeptidase inhibitor agents or derivatives or salts thereof. Aminopeptidases are widely distributed in eukaryotes and prokaryotes and catalyze the cleavage of amino acids from the amino terminus of a protein (N-terminus) or peptide substrates. Non-limiting examples of M1 aminopeptidase inhibitors suitable for used herein can be Puromycin aminonucleoside, Bestatin, Bestatin hydrochloride, Bestatin trifluoroacetate, Tosedostat, Actinonin, DG051, TNP-470, HFI-142, amastatin hydrochloride, NGR peptide Trifluoroacetate, Arphamenine A, Ebelactone A, Epiamastatin hydrochloride, L-Glutamic acid gamma-(7-amido-4-methylcoumarin), or other M1 aminopeptidase inhibitor, or a combination thereof. In some embodiments, aminopeptidase inhibitors suitable for compositions and uses disclosed herein can include, but is not limited to, inhibitors of at least one zinc-dependent aminopeptidase. In some embodiments, aminopeptidase inhibitors suitable for used herein can include, but is not limited to, inhibitors of at least one member of the M1 family of aminopeptidases. In some embodiments, M1 aminopeptidase inhibitors suitable for used herein can include, but is not limited to, inhibitors of a puromycin-sensitive aminopeptidase (NPEPPS). Puromycin-sensitive amino peptidase (are also known as cytosol alanyl aminopeptidase and/or alanine aminopeptidase (AAP)) is a zinc metallopeptidase which hydrolyzes amino acids from the N-terminus of its substrate and, in humans, is encoded by the NPEPPS gene. In some embodiments, an inhibitor of a puromycin-sensitive aminopeptidase (NPEPPS) included in compositions contemplated herein can include tosedostat, a salt or a derivative thereof. In some embodiments, concentrations of M1 aminopeptidase inhibitors of use in combination therapies can include about 1 mg per day to about 200 mg per day, about 5 mg per day to about 150 mg per day, or about 10 mg per day to about 120 mg per day. In some embodiments, concentrations of M1 aminopeptidase inhibitors of use in combination therapies can include about 1 mg, about 5 mg, about 10 mg, about 15 mg, about 20 mg, about 30 mg, about 40 mg, about 60 mg, about 80 mg, about 100 mg, about 120 mg, about 140 mg, about 160 mg, about 180 mg, or about 200 mg per day. In certain embodiments, aminopeptidase inhibitors can be administered to a subject alone or in combination with a chemotherapeutic agent, more than once per day, daily, every other day, twice weekly, three times per week, every other week, weekly or monthly or other suitable dosing regimen.

In some embodiments, other compositions which can be administered alone or in combination with M1 aminopeptidase inhibitor-containing compositions can include a platinum-based chemotherapeutic. As used herein, a “platinum-based chemotherapeutic” refers to a chemotherapeutic that is an organic compound which contains platinum as an integral part of the molecule. In some embodiments, compositions of use herein can contain one or more platinum-based chemotherapeutic agents including, but not limited to, cisplatin, carboplatin, nedaplatin, triplatin tetranitrate, phenanthriplatin, picoplatin, satraplatin, other platinum-based agent or a combination thereof.

In certain embodiments, a platinum-based chemotherapeutic can be administered to a subject simultaneously, sequentially or separately from an M1 aminopeptidase inhibitor or a derivative thereof. In some embodiments, compositions containing a platinum-based chemotherapeutic agent or salt thereof or derivative thereof can include a concentration of the platinum-based chemotherapeutic agent of about 1 mg/ml to about 100 mg/ml; about 1 mg/ml to about 90 mg/ml or about 5 mg/ml, about 10 mg/ml, about 20 mg/ml, about 30 mg/ml, about 40 mg/ml, about 50 mg/ml, about 60 mg/ml, about 80 mg/ml, about 100 mg/ml or more as determined by a health professional. In certain embodiments, the platinum-based chemotherapeutic agent or salt thereof or derivative thereof can include carboplatin. In certain embodiments, platinum-based chemotherapeutic agents can be administered to a subject alone in a chemotherapeutic cocktail (e.g., more than one chemotherapeutic agent) or in combination with at least one M1 aminopeptidase inhibitor, daily, every other day, twice weekly, every other day, every other week, weekly or monthly or other suitable dosing regimen.

In some embodiments, compositions containing agents disclosed herein can be pharmaceutical compositions. In other embodiments, compositions disclosed herein can include one or more M1 aminopeptidase inhibitors and/or one or more platinum-based chemotherapeutics. As used herein, terms “platinum-based chemotherapeutics” and “platinum-based chemotherapeutic agents,” refer to any platinum compounds and/or platinum-based antineoplastic drugs (informally called platins) used as chemotherapeutic agents to treat cancer and are used interchangeably throughout the present disclosure. In certain embodiments, these agents can be combined with a pharmaceutically acceptable carrier (excipient) to form a pharmaceutical composition for use in treating tumors in a subject.

Pharmaceutical compositions used in the present compositions and methods can include pharmaceutically acceptable carriers, excipients, or stabilizers in the form of lyophilized formulations or aqueous solutions. Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG). In some embodiments, pharmaceutical compositions described herein can be liposomes containing the one or more aminopeptidase inhibitors and/or one or more platinum-based chemotherapeutics can be prepared by methods known in the art. In some embodiments, pharmaceutical compositions herein can be liposomes with enhanced circulation time. In some embodiments, liposomes herein can be generated by the reverse phase evaporation method with a lipid composition comprising phosphatidylcholine, cholesterol and PEG-derivatized phosphatidylethanolamine (PEG-PE). Liposomes can be extruded through filters of defined pore size to yield liposomes with the desired diameter.

In some embodiments, the one or more M1 aminopeptidase inhibitors and/or one or more platinum-based chemotherapeutics disclosed herein can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions or slow-release or timed-release microparticle formulation.

In other embodiments, the pharmaceutical compositions described herein can be formulated in sustained-release format. Suitable examples of sustained-release preparations include semipermeable matrices of solid hydrophobic polymers containing the one or more aminopeptidase inhibitors and/or one or more platinum-based chemotherapeutics, which matrices are in the form of shaped articles, e.g. films, or microcapsules. Non-limiting examples of sustained-release matrices can include polyesters, hydrogels (for example, poly(2-hydroxyethyl-methacrylate), or poly(vinyl alcohol)), polylactides, copolymers of L-glutamic acid and 7 ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), sucrose acetate isobutyrate, and poly-D-(−)-3-hydroxybutyric acid.

In some embodiments, pharmaceutical compositions to be used for in vivo administration can be sterile. This is readily accomplished by, for example, filtration through sterile filtration membranes. Therapeutic pharmaceutical compositions can generally be placed into a container having a sterile access port, for example, an intravenous solution bag or vial having a stopper pierceable by a hypodermic injection needle.

In other embodiments, pharmaceutical compositions described herein can be in unit dosage forms such as tablets, pills, capsules, powders, granules, solutions or suspensions, or suppositories, for oral, parenteral or rectal administration, or administration by inhalation or insufflation. For preparing solid compositions such as tablets, the principal active ingredient (e.g., the one or more aminopeptidase inhibitors and/or one or more platinum-based chemotherapeutics) can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition can be readily subdivided into equally effective unit dosage forms such as tablets, pills and capsules. This solid preformulation composition can then subdivided into unit dosage forms of the type described above containing from about 0.1 to about 500 mg of the active ingredient (e.g., the one or more aminopeptidase inhibitors and/or one or more platinum-based chemotherapeutics) of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents (surfactant) include, but are not limited to, non-ionic agents, such as polyoxyethylenesorbitans (e.g., Tween 20, 40, 60, 80 or 85) and other sorbitans (e.g., Span 20, 40, 60, 80 or 85). Compositions with a surface-active agent are conveniently comprise between 0.05 and 5% surface-active agent and can be between about 0.1 and about 2.5%. In some embodiments, other ingredients can be added, for example mannitol or other pharmaceutically acceptable vehicles, if necessary.

In some embodiments, pharmaceutical compositions disclosed herein can be in the form of emulsions. In certain embodiments, emulsions herein can be prepared using commercially available fat emulsions, such as Intralipid, Liposyn, Infonutrol, Lipofundin and Lipiphysan. The active ingredient (e.g., the one or more M1 aminopeptidase inhibitors and/or one or more platinum-based chemotherapeutics) can be either dissolved in a pre-mixed emulsion composition or alternatively it can be dissolved in an oil (e.g., soybean oil, safflower oil, cottonseed oil, sesame oil, corn oil or almond oil) and an emulsion formed upon mixing with a phospholipid (e.g., phospholipids, soybean phospholipids or soybean lecithin) and water. In some embodiments, other ingredients can be added to the compositions herein, for example glycerol or glucose, to adjust the tonicity of the emulsion. In some embodiments, emulsions can contain up to about 20% oil, for example, between about 5% and about 20%. In some embodiments, emulsions can contain fat droplets between about 0.1 and about 1.0 μm, particularly about 0.1 and about 0.5 μm, and have a pH in the range of about 5.5 to about 8.0.

In certain embodiments, compositions disclosed herein can treat and/or prevent cancer in a subject in need. In some embodiments, compositions disclosed herein can impair tumor growth compared to tumor growth in an untreated subject with identical disease condition and predicted outcome. In some embodiments, tumor growth can be stopped following treatment with compositions disclosed herein. In other embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other embodiments, tumors treated in the subject using a composition of the present disclosure can have tumors that grow at least about 5% less (or more as described above) when compared to an untreated subject with identical disease condition and predicted outcome. In some embodiments, tumor growth or expansion can be reduced by at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, about 100% or 100% compared to a subject not treated with the compositions and methods disclosed herein having a similar condition as the treated subject.

In some embodiments, treatment of tumors with compositions or dosing regimens disclosed herein can result in shrinking of a tumor in comparison to the starting size of the tumor in a subject. In some embodiments, a tumor treated with compositions disclosed herein can shrink by about 5% or greater, or by about 10% or greater, or about 15% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater or about 100% or 100% (where the tumor is eradicated and no tumor cells remain from the treated tumor) compared to the measured starting size of the tumor. In certain embodiments, tumors of a subject can be assessed (e.g., measured) in response to a treatment regimen disclosed herein in order to modify the treatment regimen or maintain the current regimen depending on outcome at the time of evaluation.

In other embodiments, compositions disclosed herein can improve life expectancy of a subject compared to the life expectancy of an untreated subject with a similar condition as the subject being treated and/or in consideration of predicted outcome. As used herein, “life expectancy of a subject” can refer to the time at which 50 percent of subjects are alive and 50 percent have passed away. In some embodiments, life expectancy can be indefinite following treatment with a composition disclosed herein. In other embodiments, life expectancy can be increased at least about 5% or greater up to 100%, or at least about 10% or greater, at least about 20% or greater, at least about 40% or greater compared to an untreated subject with a similar health condition and predicted outcome.

In some embodiments, the methods of the present disclosure can increase anti-tumor activity of an agent or restore anti-tumor activity of an agent disclosed herein (e.g., reduce cell proliferation, tumor growth, tumor volume, tumor expansion, reduce metastasis of a tumor and/or tumor burden or load or reduce the number of metastatic lesions over time) by at least about 5% up to 100%, or about 10%, or about 20%, or about 25%, or about 30%, or about 40%, or about 50%, or about 60%, or about 70%, or about 75%, or about 80%, or about 85%, or about 90%, or about 95%, or about 99% or more as compared to tumor presence prior to treatment or in a control untreated subject. In some embodiments, reduction is measured by comparing cell proliferation, tumor growth, tumor migration and/or tumor volume in a subject before, during and/or after administration of a composition disclosed herein. In some embodiments, the method of treating or ameliorating cancer in a subject can allow one or more symptoms of the cancer to improve by at least about 10%, 20%, 30%, 40%, 50%, 60%, 70%, 80%, 90%, 95%, or more. In some embodiments, before, during, and after the administration of a composition disclosed herein, cancerous cells and/or biomarkers in a subject are measured. In accordance with these embodiments, samples can be obtained from a subject. Samples can include, but are not limited to any biological sample, such as blood, serum, plasma, urine, peritoneal fluid, and/or a biopsy from a tissue or organ. In some embodiments, the methods include administration of a composition to reduce tumor volume, size, load, and/or burden in a subject to an undetectable size, or at least to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, the methods can include administration of a compositions disclosed herein to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

In some embodiments, a subject to be treated by any compositions and/or methods disclosed herein can present with one or more cancerous solid tumors, metastatic nodes, of a combination thereof. In some embodiments, a subject can have a cancerous tumor cell source that can be less than about 0.2 cm³ to at least about 20 cm³ or larger, or at least about 6 cm³ to at least about 18 cm³ or larger.

In certain embodiments, the compositions disclosed herein can be effective for treating at least one tumor cell in a solid tumor from a subject in need. In some embodiments, the number of viable tumor cells can be reduced by at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, up to 100% compared to an untreated or unexposed tumor cells.

In certain embodiments, the compositions disclosed herein can reduce the number of viable tumor cells by about 5% up to 100%, about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, at up to 100% compared to untreated tumor cells in a subject.

Conventional methods, known to those of ordinary skill in the art of medicine, can be used to administer the compositions disclosed herein to a subject, depending upon the type of disease to be treated or the site of the disease. In some embodiments, compositions disclosed herein can be administered to a subject by intravenous infusion by subcutaneous administration, by inhalation, by intranasal administration, by patch or microparticles, or other mode of administration. In some embodiments, compositions herein can be administered to a subject orally.

In some embodiments, any of the methods disclosed herein can further include monitoring methods to assess the occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the methods disclosed herein can further adjusting the dose of the one or more M1 aminopeptidase inhibitor, the dose of one or more platinum-based chemotherapeutics agents or both the one or more M1 aminopeptidase inhibitor and the dose of one or more platinum-based chemotherapeutics agents depending on the adverse effect or effects in the subject. For example, when a moderate to severe hepatic impairment is observed in a subject after administration of a M1 aminopeptidase inhibitor (e.g., tosedostat) and/or a platinum-based chemotherapeutic, the concentration of or frequency of dosing with the M1 aminopeptidase inhibitor (e.g., tosedostat) and/or a platinum-based chemotherapeutic can be reduced.

In some embodiments, the one or more M1 aminopeptidase inhibitor can be administered concurrently with the one or more platinum-based chemotherapeutic by the same or different modes of administration. In some embodiments, the one or more M1 aminopeptidase inhibitor can be administered before, during, or after the one or more chemotherapeutics. In other embodiments, the one or more chemotherapeutics can be administered systemically. In certain embodiments, the one or more platinum-based chemotherapeutic can be administered locally directly to one or more tumors in the subject. In some embodiments, the one or more platinum-based chemotherapeutic can be administered by intravenous administration, e.g., as a bolus or by continuous infusion over a predetermined period of time, by intramuscular, intraperitoneal, intracerebrospinal, subcutaneous, intra-arterial, intra-articular, intrasynovial, intrathecal, intratumoral, oral, inhalation or topical routes. In other embodiments, the one or more platinum-based chemotherapeutic can be administered to the subject by intravenous infusion.

An effective amount of the pharmaceutical composition described herein can be administered to a subject (e.g., a human) in need of the treatment via a suitable route, systemically or locally. In some embodiments, the one or more M1 aminopeptidase inhibitor can be administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In some embodiments, the one or more M1 aminopeptidase inhibitors can be administered orally.

In some embodiments, dosages for the one or more aminopeptidase inhibitors and/or one or more platinum-based chemotherapeutics as described herein are determined empirically in subjects who have been given one or more administration(s) of the one or more aminopeptidase inhibitors and/or one or more platinum-based chemotherapeutics. In some embodiments, a subject can be administered incremental doses of the one or more aminopeptidase inhibitors and/or one or more platinum-based chemotherapeutics and effect of the agents assessed on the health of the subject and effect on tumor growth and expansion. To assess efficacy of the one or more aminopeptidase inhibitors and/or one or more platinum-based chemotherapeutics, an indicator of the disease/disorder can be followed (e.g., number of tumors, size of tumors, gene expression in tumor cells, and the like).

In some embodiments, methods disclosed herein for treating cancer in a subject with one or more M1 aminopeptidase inhibitors and/or one or more platinum-based chemotherapeutics can further include treating the subject with at least one additional anti-cancer therapy, for example, other chemotherapy, radiotherapy, immunotherapy, or surgery. In some embodiments, other anti-cancer therapies administered to the subject can include, but are not limited to, one or more of gemcitabine, methotrexate, vinblastine, and/or adriamycin. In other embodiments, compositions disclosed herein can be administered to a subject who has completed at least one other anti-cancer therapy or who is currently undergoing at least one other anti-cancer therapy. In some embodiments, compositions disclosed herein can be administered to a subject having received and/or currently receiving at least one immune-modulatory agent. Non-limiting examples of such immune-modulatory agents include, but are not limited to, anti-PD1, anti-PD-L1, anti-CTLA-4, anti-OX40, anti-CD137, etc. Non-limiting examples of PD-1 inhibitors include, but are not limited to, anti-PD-1 antibodies, such as pembrolizumab, nivolumab, and cemiplimab. Non-limiting examples of PD-L1 inhibitors can include atezolizumab, durvalumab, and avelumab. A non-limiting example of a CTLA-4 inhibitor is the anti-CTLA-4 antibody ipilimumab. In some embodiments, an immunomodulatory agent can be one or more inhibitors that target a checkpoint molecule selected from CD40, GITR, LAG-3, OX40, TIGIT and TIM-3. In some embodiments, the additional one or more chemotherapeutics can include an antimetabolite, a microtubule inhibitor, or a combination thereof. Antimetabolites can include, for example, folic acid antagonist (e.g., methotrexate) and nucleotide analogs such as pyrimidine antagonist (e.g., 5-fluorouracil, foxuridine, cytarabine, capecitabine, and gemcitabine), purine antagonist (e.g., 6-mercaptopurine and 6-thioguanine), and adenosine deaminase inhibitor (e.g., cladribine, fludarabine and pentostatin).

In some embodiments, methods of treatment including compositions of at least one M1 aminopeptidase inhibitor and/or one or more platinum-based chemotherapeutic can depend on the cancer type, grade of cancer, stage or cancer or a combination thereof. In certain embodiments, methods of treatment including compositions of at least one M1 aminopeptidase inhibitor and/or one or more platinum-based chemotherapeutic can depend on the stage of cancer as determined by the TNM system wherein “T” stands for tumor, “N” stands for node, and “M” stands for metastasis. When applying the TNM system, the following are considered: Tumor (T)—How large is the primary tumor? Where is it located?; Node (N)—Has the tumor spread to the lymph nodes? If so, where and how many nodes are involved?; and Metastasis (M)—Has the cancer spread to other parts of the body, affected other organs? If so, where and how severe? In some embodiments, methods of treatment including compositions of at least one aminopeptidase inhibitor and/or one or more platinum-based chemotherapeutic can depend on the stage of bladder cancer. One of skill in the art (e.g., a physician) can assign the stage of the bladder cancer in a subject by combining the T, N, and M classifications or other factors. The stages of bladder cancer contemplated and as used herein are provided in Table 1.

TABLE 1

| Bladder Cancer Stage | Description |
|---|---|
| Stage 0a | This is an early cancer that is only found on the surface of the inner |
| | lining of the bladder. Cancer cells are grouped together and can often be |
| | easily removed. The cancer has not invaded the muscle or connective |
| | tissue of the bladder wall. This type of bladder cancer is also called |
| | noninvasive papillary urothelial carcinoma (Ta, N0, M0). |
| Stage 0is | This stage of cancer, also known as a flat tumor or carcinoma in situ |
| | (CIS), is found only on the inner lining of the bladder. It has not grown |
| | in toward the hollow part of the bladder, and it has not spread to the |
| | thick layer of muscle or connective tissue of the bladder (Tis, N0, M0). |
| | This is always a high-grade cancer and is considered an aggressive |
| | disease because it can often lead to muscle-invasive disease. |
| Stage I | The cancer has grown through the inner lining of the bladder and into the |
| | lamina propria. It has not spread to the thick layer of muscle in the |
| | bladder wall or to lymph nodes or other organs (T1, N0, M0). |
| Stage II | The cancer has spread into the thick muscle wall of the bladder. It is also |
| | called invasive cancer or muscle-invasive cancer. The tumor has not |
| | reached the fatty tissue surrounding the bladder and has not spread to the |
| | lymph nodes or other organs (T2, N0, M0). |
| Stage III | The cancer has spread throughout the muscle wall to the fatty layer of |
| | tissue surrounding the bladder (perivesical tissue) or to the prostate in a |
| | man or the uterus and vagina in a woman. Or, the cancer has spread to |
| | the regional lymph nodes. |
| Stage IIIA | The tumor has grown into the perivesical tissue or has spread to the |
| | prostate, uterus, or vagina, but has not spread to the lymph nodes or |
| | other organs (T3a, T3b, or T4a; N0; M0), or the cancer has spread to a |
| | single regional lymph node (T1 to T4a, N1, M0). |
| Stage IIIB | The cancer has spread to 2 or more regional lymph nodes or to the |
| | common iliac lymph nodes (T1 to T4a, N2 or N3, M0). |
| Stage IV | The tumor has spread into the pelvic wall or abdominal wall, or the |
| | cancer has spread to lymph nodes outside of the pelvis or to other parts of |
| | the body. |
| Stage IVA | The tumor has spread to the pelvic wall or the abdominal wall but not |
| | to other parts of the body (T4b, any N, M0), or the cancer has spread |
| | to lymph nodes located outside of the pelvis (any T, any N, M1a). |
| Stage IVB | The cancer has spread other parts of the body (any T, any N, M1b). |

As used in Table 1, “grade” describes how much cancer cells look like healthy cells when viewed under a microscope. Healthy tissue usually contains many different types of cells grouped together. If the cancer looks similar to healthy tissue and has different cell groupings, it is called “differentiated” or a “low-grade tumor.” If the cancerous tissue looks very different from healthy tissue, it is called “poorly differentiated” or a “high-grade tumor.”

In some embodiments, compositions and methods disclosed herein can be used to treat a Stage I, Stage II, Stage III, or Stage IV bladder cancer in a subject. In other embodiments, compositions and methods disclosed herein can be used to treat a Stage II, III, or IV bladder cancer.

In some embodiments, methods for the measuring NPEPPS levels or concentrations in a targeted tumor are disclosed. In accordance with these embodiments, NPEPPS can be used as at least one biomarker, for example, to select or identify patients or subjects in need of an aminopeptidase treatment. In certain embodiments, NPEPPS levels can determine tumor burden, disease progression or projected response to treatment.

In some embodiments, a combination of biomarkers can be used as indicators of a process, event or condition. In other embodiments, a combination of biomarkers can be used as indicators of a process, event or condition. In certain embodiments, a combination of biomarkers used as indicators of a process, event or condition can include at least two biomarkers including, but not limited to, NPEPPS, at least one subunit of volume-regulated anion channel (VRAC) (e.g., LRRC8A, LRRC8B, LRRC8C, LRRC8D, LRRC8E), or any combination thereof. In other embodiments, a combination of biomarkers used as indicators of a process, event or condition can include at least two biomarkers up to about 50 biomarkers (e.g., about 2, 4, 6, 8, 10, 12, 14, 16, 18, 20, 25, 30, 35, 40, 45, or 50). In some embodiments, a combination of biomarkers used as indicators of a process, event or condition can include at least two biomarkers to about 50 biomarkers (e.g., about 2, 4, 6, 8, 10, 12, 14, 16, 18, 20, 25, 30, 35, 40, 45, or 50) wherein one of the biomarkers can include NPEPPS, at least one subunit of VRAC (e.g., LRRC8A, LRRC8B, LRRC8C, LRRC8D, LRRC8E), or any combination thereof.

As used herein, “predictive biomarker” can refer to a biomarker that can be used in advance of therapy to estimate the likelihood or predictability of response to a given therapeutic agent or class of therapeutic agents. In some embodiments, a combination of predictive biomarkers can be used in advance of therapy to estimate the likelihood or predictability of response to a given therapeutic agent or class of therapeutic agents. In other embodiments, a combination of predictive biomarkers used in advance of therapy to estimate the likelihood or predictability of response to a given therapeutic agent or class of therapeutic agents can have at least two predictive biomarkers. In some embodiments, a combination of predictive biomarkers used in advance of therapy to estimate the likelihood or predictability of response to a given therapeutic agent or class of therapeutic agents can have at least two predictive biomarkers where one of the predictive biomarkers can be NPEPPS, at least one subunit of VRAC (e.g., LRRC8A, LRRC8B, LRRC8C, LRRC8D, LRRC8E), or any combination thereof. In some embodiments, a combination of predictive biomarkers used in advance of therapy to estimate the likelihood or predictability of response to a given therapeutic agent or class of therapeutic agents can include two predictive biomarkers to about 50 predictive biomarkers (e.g., about 2, 4, 6, 8, 10, 12, 14, 16, 18, 20, 25, 30, 35, 40, 45, or 50) where one of the predictive biomarkers of the combination can include NPEPPS, at least one subunit of VRAC (e.g., LRRC8A, LRRC8B, LRRC8C, LRRC8D, LRRC8E), or any combination thereof.

In certain embodiments, a therapeutic agent can include, one or more M1 aminopeptidase inhibitor and/or one or more platinum-based chemotherapeutics. In some embodiments, the predictive biomarker can include measuring serum or plasma NPEPPS, LRRC8A, LRRC8B, LRRC8C, LRRC8D, and/or LRRC8E concentration. In other embodiments, the predictive biomarker can include measuring NPEPPS, LRRC8A, LRRC8B, LRRC8C, LRRC8D, and/or LRRC8E levels or concentration in a tumor and/or NPEPPS, LRRC8A, LRRC8B, LRRC8C, LRRC8D, and/or LRRC8E levels or concentration in organoid cultures derived from a tumor. In certain embodiments, NPEPPS, LRRC8A, LRRC8B, LRRC8C, LRRC8D, and/or LRRC8E levels can be compared to a healthy control or healthy control sample to assess the need for aminopeptidase inhibition in a subject. In some embodiments, methods disclosed herein can be used for identifying a subject having or at risk of developing a drug-resistant cancer and/or for determining severity of the cancer in a cancer patient, based on the levels of NPEPPS, LRRC8A, LRRC8B, LRRC8C, LRRC8D, and/or LRRC8E levels measured in a biological sample as disclosed herein.

As used herein, the term “biological sample” can include any sample obtained from a subject. A suitable biological sample can be obtained from a subject as described herein by routine practice. Non-limiting examples of biological samples include fluid samples such as blood (e.g., whole blood, plasma, or serum), urine, and saliva, and solid samples such as tissue (e.g., skin, lung, or nasal) and feces. Such samples can be collected using any method known in the art or described herein, e.g., buccal swab, nasal swab, venipuncture, biopsy, urine collection, or stool collection. In some embodiments, the biological sample can be a blood sample. In some other embodiments, the blood sample is a serum sample or a plasma sample. In some embodiments, a biological sample can be derived from a tissue and/or tumor biopsy collected from the subject e.g., patient-derived organotypic tumor spheroids prepared as described herein. In some embodiments, a biological sample can be transurethral resection of a bladder tumor (TURBT).

In some embodiments, methods disclosed herein for measuring NPEPPS, LRRC8A, LRRC8B, LRRC8C, LRRC8D, and/or LRRC8E levels or concentrations in a biological sample of subject can be measured by routine practice. Methods for detecting and/or assessing levels of gene expression of or protein levels of NPEPPS, LRRC8A, LRRC8B, LRRC8C, LRRC8D, and/or LRRC8E in a biological sample are well known in the art, and all suitable methods for detecting and/or assessing levels of gene expression levels known to one of skill in the art are contemplated within the scope of the invention. In some embodiments, gene expression of NPEPPS, LRRC8A, LRRC8B, LRRC8C, LRRC8D, and/or LRRC8E in a biological sample can be measured by high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or a combination thereof. In some embodiments, gene expression of NPEPPS, LRRC8A, LRRC8B, LRRC8C, LRRC8D, and/or LRRC8E as disclosed herein can be measured by any known or future method suitable to assess gene expression.

In other embodiments, methods for detecting and/or assessing levels of protein expression (i.e., protein level) of NPEPPS, LRRC8A, LRRC8B, LRRC8C, LRRC8D, and/or LRRC8E in a biological sample are well known in the art, and all suitable methods for detecting and/or assessing levels of protein expression levels known to one of skill in the art are contemplated within the scope of the invention. In some embodiments, protein expression of NPEPPS, LRRC8A, LRRC8B, LRRC8C, LRRC8D, and/or LRRC8E in a biological sample can be measured by Western blotting, enzyme-linked immunosorbent assay (ELISA), mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), matrix-assisted laser desorption/ionization (MALDI), or a combination thereof. In some embodiments, protein expression of NPEPPS, LRRC8A, LRRC8B, LRRC8C, LRRC8D, and/or LRRC8E as disclosed herein can be measured by any known or future method suitable to assess protein expression.

In some embodiments, a subject can be a human patient having an elevated level of NPEPPS, LRRC8A, LRRC8B, LRRC8C, LRRC8D, and/or LRRC8E as relative to a control level. A control level can refer to the level of NPEPPS, LRRC8A, LRRC8B, LRRC8C, LRRC8D, and/or LRRC8E in a matched sample of a subject of the same species (e.g., human) who are free of the solid tumor. In some embodiments, the control level represents the level of NPEPPS, LRRC8A, LRRC8B, LRRC8C, LRRC8D, and/or LRRC8E in healthy subjects.

In certain embodiments, the present disclosure provides kits for use in treating or alleviating cancer in a subject described herein. For example, kits can include, but are not limited to, one or more containers including, for example, one or more M1 aminopeptidase inhibitor, e.g., any of those described herein (e.g., tosedostat) and one or more platinum-based chemotherapeutic described herein (e.g., a carboplatin). In other embodiments, kits can include a control NPEPPS, LRRC8A, LRRC8B, LRRC8C, LRRC8D, and/or LRRC8E sample for assessing level of NPEPPS, LRRC8A, LRRC8B, LRRC8C, LRRC8D, and/or LRRC8E in a sample from a subject.

In some embodiments, kits disclosed herein can include instructions for use in accordance with any of the methods described herein. The included instructions can have a description of administration of the M1 aminopeptidase inhibitor(s), and the one or more platinum-based chemotherapeutics, to treat, delay the onset, or alleviate a target disease as those described herein. In some embodiments, the kit can further include a description of selecting an individual suitable for treatment based on identifying whether that individual has the target disease, e.g., applying the diagnostic method as described herein. In still other embodiments, the instructions can have a description of administering any one of the compositions described herein to an individual at risk of the target disease.

In other embodiments, kit instructions relating to the use of one or more aminopeptidase inhibitors (e.g., tosedostat) and one or more platinum-based chemotherapeutic described herein (e.g., a carboplatin) can generally include information as to dosage, dosing schedule, and route of administration for the intended treatment. The containers can be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the solid tumor. In some embodiments, instructions are provided for practicing any of the methods described herein. In other embodiments, instructions are provided for assessing NPEPPS, LRRC8A, LRRC8B, LRRC8C, LRRC8D, and/or LRRC8E as a biomarker of disease (e.g., late-stage cancer).

In certain embodiments, kits disclosed herein have suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container can be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is at least one M1 aminopeptidase inhibitor (e.g., tosedostat) as those described herein.

In some embodiments, kits herein can optionally provide additional components such as buffers and interpretive information. Normally, the kit comprises a container and a label or package insert(s) on or associated with the container. In some embodiments, the invention provides articles of manufacture comprising contents of the kits described above.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

Human Cell Lines Derived from Muscle-Invasive Bladder Cancer

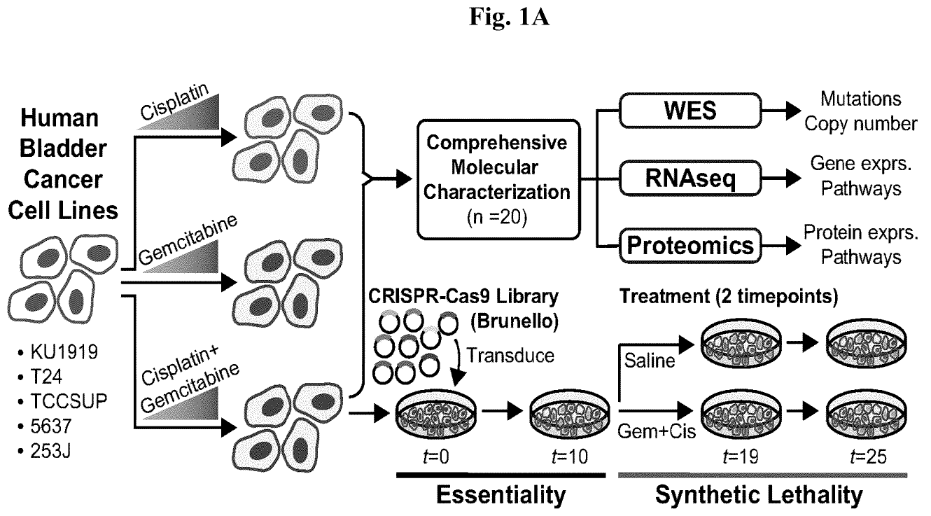

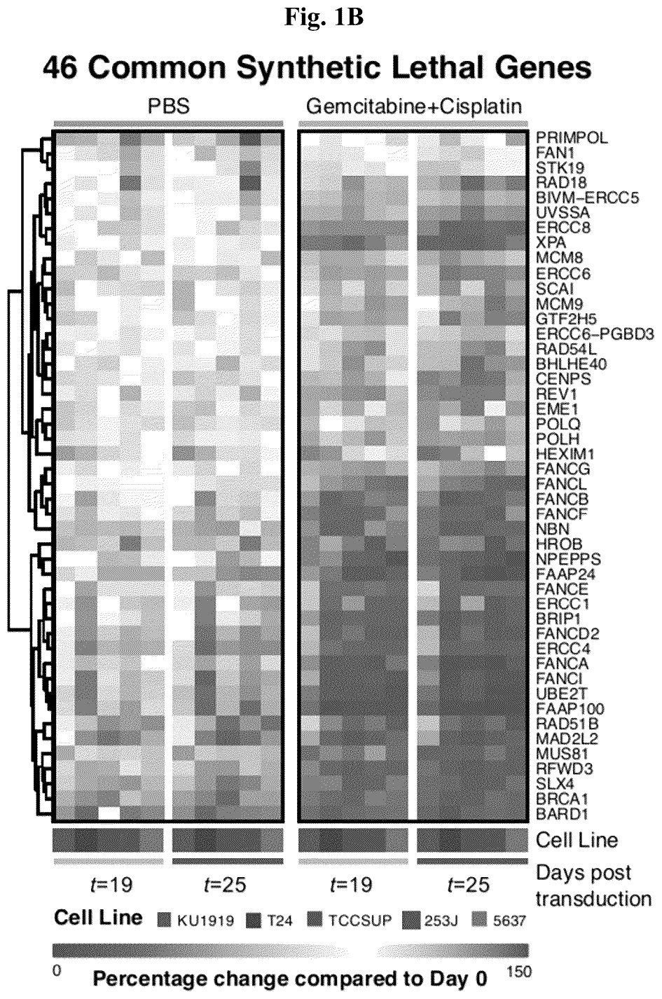

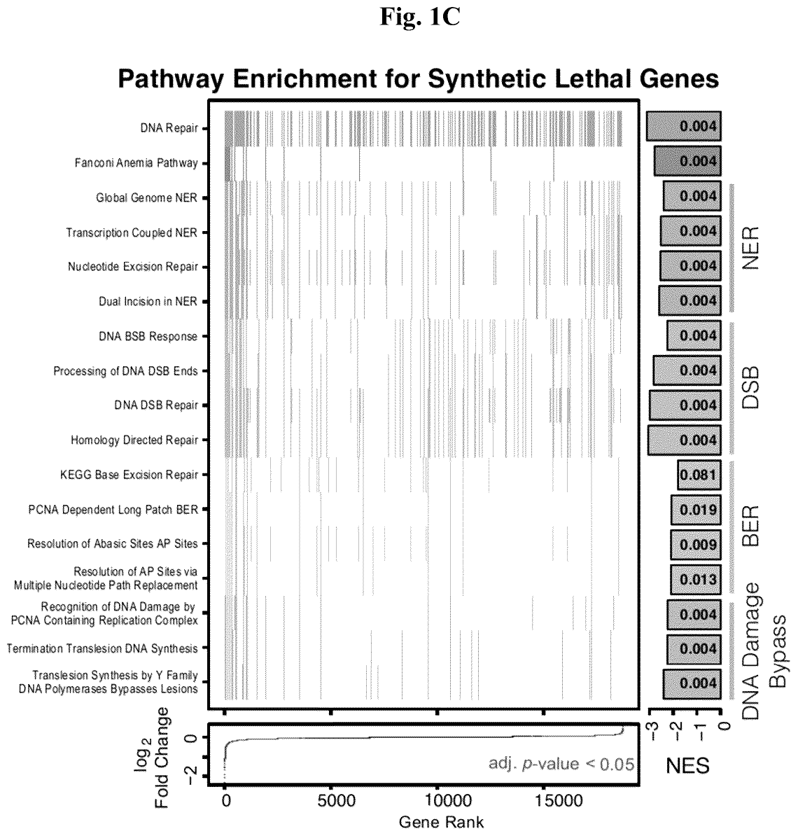

In one exemplary method, to study acquired gemcitabine plus cisplatin chemotherapy resistance in human bladder cancer (BCa), cell lines derived from muscle-invasive bladder cancer (MIBC) patients compiled in the Resistant Cancer Cell Line (RCCL) collection were used. A total of 5 MIBC cell lines, KU1919, 5637, T24, TCCSUP, and 253J, were available with matched sets of the parental cell line (-Par) and the derivatives that had been made resistant through dose escalation to cisplatin (-Cis), gemcitabine (-Gem), and gemcitabine plus cisplatin (-GemCis) concurrently (FIG. 1A; Table 2).

TABLE 2

| | Derivative Cell Lines | | |
|---|---|---|---|
| Parental | | | Gemcitabine/ |
| Cell | Cisplatin (-Cis) | Gemcitabine (-Gem) | Cisplatin (-GemCis) |
| Line | (ng/mL) | (ng/mL) | (ng/mL) |
| T 24 | 1000 | 20 | 20/1000 |
| TCC-SUP | 1000 | 10 | 20/1000 |
| 5637 | 1000 | 20 | 20/1000 |
| KU-19-19 | 1000 | 20 | 20/2000 |
| 253J | 1000 | 20 | 20/1000 |

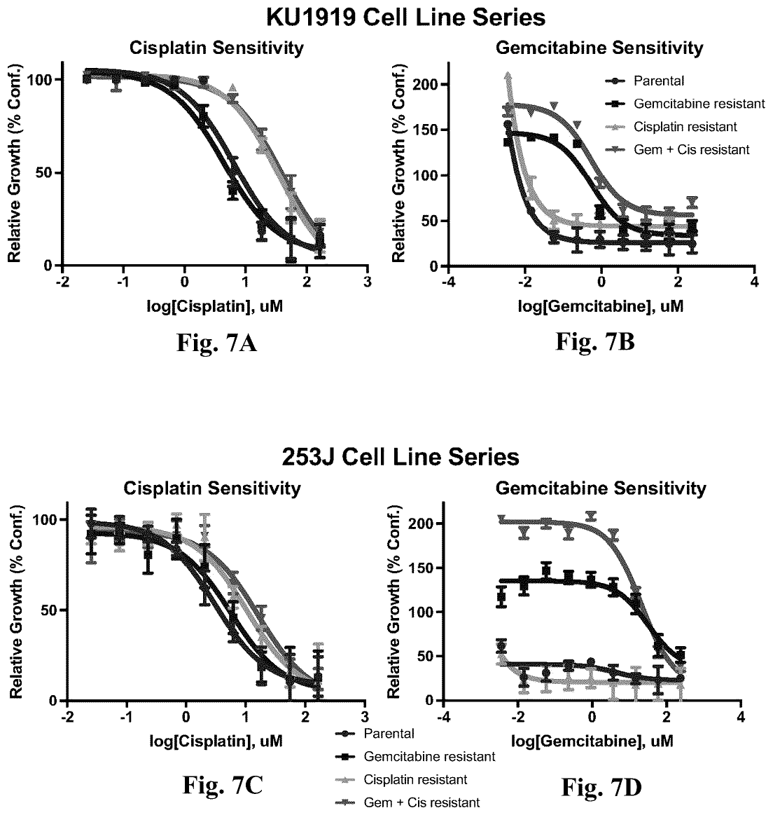

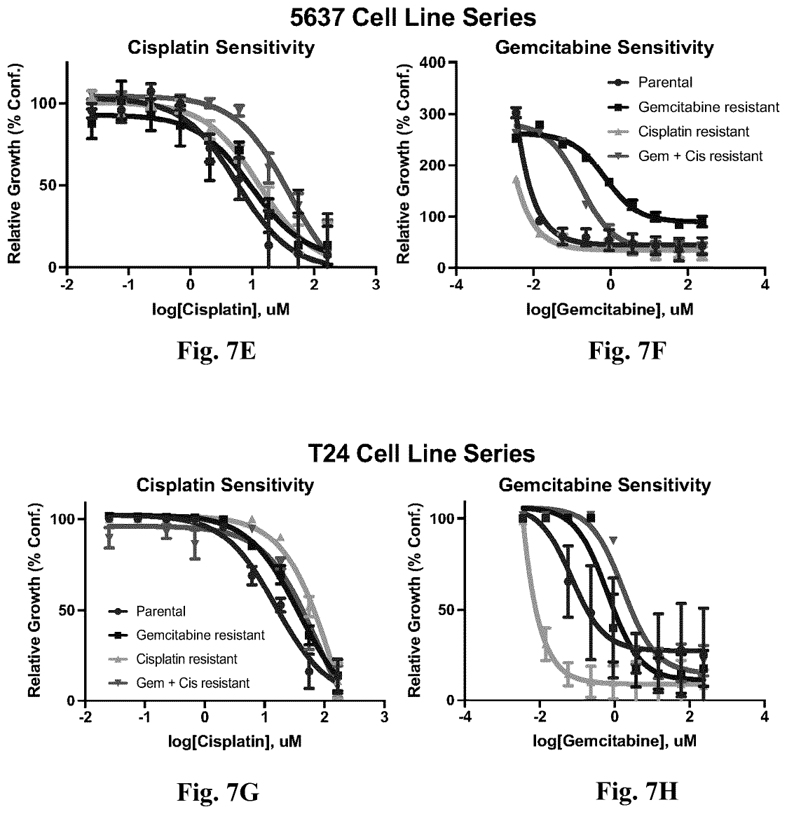

Resistance to the associated drugs was confirmed for all resistant derivatives in comparison to the parental lines; the drug concentrations were stable and consistent with those reported in the RCCL (FIGS. 7A-7J).

These 5 human BCa cell lines represent a range of common clinicopathologic characteristics and genetic drivers seen in MIBC (Table 3). These cells were derived from 3 females (T24, TCCSUP, 253J) and 2 males (5637, KU1919), across a range of stages and grades, and represented both luminal (5637) and basal (T24, TCCSUP, 253J) subtypes. These cells also contained alterations in putative BCa drivers as reported in TCGA and pathogenic mutations reported in ClinVar.

TABLE 3

| Feature | KU1919 | T24 | TCCSUP | 5637 | 253J |
|---|---|---|---|---|---|
| Sex | Male | Female | Female | Male | Female |
| Stage | T3 | Ta | N/A | N/A | T4 |
| Grade | G3 | G3 | G4 | G2 | G4 |
| Base47 | N/A | Basal | Basal | Luminal | Basal |
| Subtype | | | | | |
| TP53 | | Y126X | E349X | | |
| HRAS | | G12V | | | |
| NRAS | Q61R | | | | |
| PIK3CA | | | E545K | | E545G |
| TERT | | | | | |
| ARID1A | Y1052X | | | | |
| KMT2D | T2441Pfs*44 | | | Q2813X | |
| KDM6A | Q915X | | | | |
| FAT1 | | S2682X | D1536N | | |
| KMT2C | | R4225X; | | | |
| | | A3559T | | | |
| ERBB2 | | | | S310F | |
| ERBB3 | | E1219K | | | |
| EP300 | | C1201Y | | | |
| FBXW7 | | | S66X | | |
| ASXL2 | | | E330Q | | |
| ATM | | | | H1876Q | |
| RB1 | | | | Y325X | |
| AKT1 | E17K | | | | |
| RYR2 | | R2401H | | | |
| NFE2L2 | | | | | G81S |

### Example 2

### Genome-Wide CRISPR Screens Identify 46 Common Synthetic Lethal Genes

To study the connection between drug resistance and genes, whole-genome loss of function screens was performed in each of the 5 GemCis-resistant cell line derivatives. After transduction of the Brunello, pooled CRISPR knockout library, the cells were passaged for 10 days to clear essential genes, then split into a saline (PBS) vehicle control treatment group or a gemcitabine plus cisplatin combination treatment group (FIG. 1A). Each screen was performed at the established drug concentrations that allowed the GemCis-resistant cells to grow unrestricted, but where the same concentrations significantly inhibited the growth of the associated parental lines. CRISPR screening parameters for each cell line are reported in Table 4.

TABLE 4

| | | | | | | Cisplatin/ |
|---|---|---|---|---|---|---|
| | | | | | | Gemcitabine |
| | | | | | | concentrations |
| | | | | Minimum | Minimum | used during |
| | | | | Coverage T0 | Coverage | synthetic |
| | Lentivirus/ | | | through T10 | T10 | lethal phase |
| Cell | Plasmid | Polybrene | Puromycin | (fold coverage | through | (T10 through T25) |
| line | Batch Used | (μg/mL) | (μg/mL) | per sgRNA) | T25 | (ng/mL) |
| T24- | Batch 1 | 8 | 1 | 3220 | 3020 | 1000/20 |
| GemCis | | | | | | |
| TCCSUP- | Batch 1 | 8 | 1.5 | 2980 | 942 | 1000/20 |
| GemCis | | | | | | |
| 253J- | Batch 2 | 8 | 7 | 1050 | 491 | 1000/20 |
| GemCis | | | | | | |
| KU1919- | Batch 2 | 8 | 2.5 | 785 | 785 | 2000/20 |
| GemCis | | | | | | |
| 5637- | Batch 2 | 8 | 0.5 | 525 | 525 | 1000/20 |
| GemCis | | | | | | |

Each screen was conducted at a minimum of 500-fold coverage per sgRNA throughout the entire experiment. sgRNAs were measured at days 19 and 25, which are 9 and 15 days after the start of treatment. This screening design allowed for the identification of sgRNAs that were differentially abundant between the saline and gemcitabine plus cisplatin-treated arms at various timepoints, and to identify sgRNA that were preferentially enriched or depleted over time.

Genes were defined as “synthetic lethal” with gemcitabine plus cisplatin as those for which the combined cognate sgRNA counts were significantly lower (moderated t-test, FDR<0.05) in the gemcitabine plus cisplatin-treated arm compared to the saline arm considering measurements treating days 19 and 25 as covariates. Using these criteria, 235 synthetic lethal genes were identified for KU1919-GemCis, 888 for T24-GemCis, 2099 for TCCSUP-GemCis, 2369 for 253J-GemCis, and 511 for 5637-GemCis. Next, the overlap between these results was identified and 46 genes were found that were commonly synthetic lethal across all 5 cell lines (FIGS. 1B-1C). These 46 genes, in no particular order are provided in Table 5 below. TABLE 5

TABLE 5

| Gene | Full Gene Name | |
|---|---|---|
| Symbol | (provided by HGNC) | Previous Symbols/Previous names/Aliases |
| PRIMPOL | primase and DNA directed | CCDC111; coiled-coil domain containing 111; primase |
| | polymerase | and polymerase (DNA-directed); FLJ33167 |
| FAN1 | FANCD2 and FANCI | KIAA1018; MTMR15; KIAA1018; myotubularin |
| | associated nuclease 1 | related protein 15; FANCD2/FANCI-associated |
| | | nuclease 1 |
| STK19 | serine/threonine kinase 19 | D6S60; G11; RP1 |
| RAD18 | RAD18 E3 ubiquitin | RAD18 homolog (S. cerevisiae); RNF73 |
| | protein ligase | |
| BIVM-ERCC5 | BIVM-ERCC5 | — |
| | readthrough | |
| UVSSA | UV stimulated scaffold | KIAA1530 |
| | protein A | |
| ERCC8 | ERCC excision repair 8, | CKN1; Cockayne syndrome 1 (classical); |
| | CSA ubiquitin ligase | excision repair cross-complementing rodent repair |
| | complex subunit | deficiency, complementation group 8; xcision repair |
| | | cross-complementation group 8; CSA |
| XPA | XPA, DNA damage | xeroderma pigmentosum, complementation group A; |
| | recognition and repair | XPAC; XP1 |
| | factor | |
| MCM8 | minichromosome | C20orf154; chromosome 20 open reading frame 154; |
| | maintenance 8 | minichromosome maintenance complex component 8; |
| | homologous | MGC4816; MGC12866; MGC119522; MGC119523; |
| | recombination repair | dJ967N21.5; REC; REC homolog (Drosophila) |
| | factor | |
| ERCC6 | ERCC excision repair 6, | CKN2; excision repair cross-complementing rodent |
| | chromatin remodeling | repair deficiency, complementation group 6; excision |
| | factor | repair cross-complementation group 6; CSB; RAD26; |
| | | ARMD5; Cockayne syndrome B protein |
| SCAI | suppressor of cancer cell | C9orf126; chromosome 9 open reading frame 126; |
| | invasion | FLJ36664; NET40 |
| MCM9 | minichromosome | MCMDC1; C6orf61; minichromosome maintenance |
| | maintenance 9 | deficient domain containing 1; chromosome 6 open |
| | homologous | reading frame 61; minichromosome maintenance |
| | recombination repair | complex component 9; MGC35304; dJ329L24.3; |
| | factor | FLJ20170 |
| GTF2H5 | general transcription factor | C6orf175; TTD; chromosome 6 open reading frame |
| | IIH subunit 5 | 175; trichothiodystrophy; general transcription factor |
| | | IIH, polypeptide 5; FLJ30544; bA120J8.2; TTD-A; |
| | | TFB5; TFIIH; TTDA; DNA repair syndrome |
| | | trichothiodystrophy group A |
| ERCC6-PGBD3 | ERCC6-PGBD3 | — |
| | readthrough | |
| RAD54L | RAD54 like | RAD54 (S. cerevisiae)-like; RAD54 like (S. cerevisiae); |
| | | hHR54; hRAD54; RAD54A |
| BHLHE40 | basic helix-loop-helix | STRA13; BHLHB2; basic helix-loop-helix domain |
| | family member e40 | containing, class B, 2; basic helix-loop-helix family, |
| | | member e40; DEC1; SHARP2; Clast5; differentially |
| | | expressed in chondrocytes 1; differentiated embryo |
| | | chondrocyte expressed gene 1 |
| CENPS | centromere protein S | APITD1; MHF1; apoptosis-inducing, TAF9-like domain |
| | | 1; FANCM associated histone fold protein 1; CENP-S; |
| | | FAAP16 |
| REV1 | REV1 DNA directed | REV1L; REV1 (yeast homolog)- like; REV1-like |
| | polymerase | (yeast); REV1 homolog (S. cerevisiae); REV1, |
| | | polymerase (DNA directed) |
| EME1 | essential meiotic structure- | essential meiotic endonuclease 1 homolog 1 (S. pombe); |
| | specific endonuclease 1 | FLJ31364; MMS4L; SLX2A; SLX2 structure-specific |
| | | endonuclease subunit homolog A (S. cerevisiae) |
| POLQ | DNA polymerase theta | polymerase (DNA directed), theta; polymerase (DNA) |
| | | theta; POLH |
| POLH | DNA polymerase eta | polymerase (DNA directed), eta; polymerase (DNA) eta; |
| | | RAD30A; XP-V |
| HEXIM1 | HEXIM P-TEFb complex | hexamethylene bisacetamide inducible 1; CLP-1; HIS1; |
| | subunit 1 | MAQ1; EDG1 |
| FANCG | FA complementation | XRCC9; Fanconi anemia complementation group G; |
| | group G | FAG; DNA repair protein XRCC9; X-ray repair, |
| | | complementing defective, in Chinese hamster, 9; X-ray |
| | | repair complementing defective repair in Chinese |
| | | hamster cells 9 |
| FANCL | FA complementation | PHF9; PHD finger protein 9; Fanconi anemia |
| | group L | complementation group L; FLJ10335; FAAP43; Pog |
| FANCB | FA complementation | Fanconi anemia complementation group B; FAB; |
| | group B | FLJ34064; FAAP95 |
| FANCF | FA complementation | Fanconi anemia complementation group F; FAF |
| | group F | |
| NBN | nibrin | NBS; NBS1; Nijmegen breakage syndrome 1 (nibrin); |
| | | ATV; AT-V2; AT-V1 |
| HROB | homologous | C17orf53; chromosome 17 open reading frame 53; |
| | recombination factor with | MGC3130; MCM8IP |
| | OB-fold | |
| NPEPPS | aminopeptidase puromycin | PSA; MP100; puromycin-sensitive aminopeptidase; |
| | sensitive | metalloproteinase MP100 |
| FAAP24 | FA core complex | C19orf40; chromosome 19 open reading frame 40; |
| | associated protein 24 | Fanconi anemia core complex associated protein 24; |
| | | FLJ46828; MGC32020; Fanconi anemia-associated |
| | | protein, 24 kDa |
| FANCE | FA complementation | FACE; Fanconi anemia complementation group E; FAE |
| | group E | |
| ERCC1 | ERCC excision repair 1, | excision repair cross-complementing rodent repair |
| | endonuclease non-catalytic | deficiency, complementation group 1 (includes |
| | subunit | overlapping antisense sequence); excision repair cross- |
| | | complementation group 1; RAD10 |
| BRIP1 | BRCA1 interacting protein | OF; BACH1; FANCJ; BRCA1/BRCA2-associated |
| | C-terminal helicase 1 | helicase 1 |
| FANCD2 | FA complementation | FACD; FANCD; Fanconi anemia complementation |
| | group D2 | group D2; FAD; FA-D2 |
| ERCC4 | ERCC excision repair 4, | XPF; excision repair cross-complementing rodent repair |
| | endonuclease catalytic | deficiency, complementation group 4; excision repair |
| | subunit | cross-complementation group 4; RAD1; FANCQ; |
| | | xeroderma pigmentosum, complementation group F |
| FANCA | FA complementation | FACA; FANCH; Fanconi anemia complementation |
| | group A | group A; FAA; FA-H; FAH |
| FANCI | FA complementation | KIAA1794; KIAA1794; Fanconi anemia |
| | group I | complementation group I; FLJ10719 |
| UBE2T | ubiquitin conjugating | ubiquitin-conjugating enzyme E2T; HSPC150; FANCT |
| | enzyme E2 T | |
| FAAP100 | FA core complex | C17orf70; chromosome 17 open reading frame 70; |
| | associated protein 100 | Fanconi anemia core complex associated protein 100; |
| | | FLJ22175; Fanconi anemia-associated protein, 100 kDa |
| RAD51B | RAD51 paralog B | RAD51L1; RAD51 (S. cerevisiae)-like 1; RAD51-like 1 |
| | | (S. cerevisiae); RAD51 homolog B (S. cerevisiae); |
| | | REC2; hREC2; R51H2 |
| MAD2L2 | mitotic arrest deficient 2 | MAD2 (mitotic arrest deficient, yeast, homolog)-like 2; |
| | like 2 | MAD2B; REV7; POLZ2; FANCV; mitotic arrest |
| | | deficient homolog-like 2; polymerase (DNA-directed), |
| | | zeta 2, accessory subunit |
| MUS81 | MUS81 structure-specific | MUS81 endonuclease homolog (yeast); MUS81 |
| | endonuclease subunit | endonuclease homolog (S. cerevisiae); FLJ44872; |
| | | SLX3; SLX3 structure-specific endonuclease subunit |
| | | homolog (S. cerevisiae) |
| RFWD3 | ring finger and WD repeat | FLJ10520; RNF201; FANCW |
| | domain 3 | |
| SLX4 | SLX4 structure-specific | BTBD12; BTB (POZ) domain containing 12; SLX4 |
| | endonuclease subunit | structure-specific endonuclease subunit homolog (S. |
| | | cerevisiae); KIAA1784; KIAA1987; FANCP; Fanconi |
| | | anemia, complementation group P |
| BRCA1 | BRCA1 DNA repair | breast cancer 1, early onset; breast cancer 1; RNF53; |
| | associated | BRCC1; PPP1R53; FANCS; BRCA1/BRCA2- |
| | | containing complex, subunit 1; protein phosphatase 1, |
| | | regulatory subunit 53; Fanconi anemia, |
| | | complementation group S |
| BARD1 | BRCA1 associated RING | — |
| | domain 1 | |

The patterns of synthetic lethality varied across the experiment, with some genes increasing cell growth in control, saline treatment, then reducing growth in gemcitabine plus cisplatin treatment (FIG. 1B; PRIMPOL; FAN1; STK19; RAD18; BIVM-ERCC5; UVSSA; ERCC8; XPA; MCM8; ERCC6; SCAI). Other genes had very little impact on cell growth in control, saline treatment, but then reduced growth when treated with gemcitabine plus cisplatin (FIG. 1B, MCM9; GTF2H5; ERCC6-PGBD3; RAD54L; BHLHE40; CENPS; REV1; EME1, POLQ; POLH; HEXIM1; FANCG; FANCL; FANCB; FANCF; NBN; HROB; NPEPPS).

Finally, some genes reduced cell growth in control, saline treatment, and further reduced growth with gemcitabine plus cisplatin treatment (FIG. 1B; FAAP24; FANCE; ERCC1; BRIP1; FANCD2; ERCC4; FANCA; FANCI; UBE2T; FAAP100; RAD51B; MAD21L2; MUS81; RFWD3; SLX4; BRCA1; BARD 1). Overall, the data supported that these 46 genes commonly and significantly reduced growth of the GemCis-resistant cells when treated with gemcitabine plus cisplatin.

To identify the most robust and commonly synthetic lethal candidate genes, gene set enrichment analysis was performed on the full list of genes ranked according to their synthetic lethality combined over all 5 cell lines. Of the 46 commonly synthetic lethal genes, and illustrated in FIG. 1B, some increased cell growth in PBS treatment, then reduced growth in gemcitabine plus cisplatin treatment. Other genes had very little impact on cell growth in PBS treatment, but then reduced growth when treated with gemcitabine plus cisplatin. Finally, some genes reduced cell growth in PBS treatment and further reduced growth with gemcitabine plus cisplatin treatment. Of the 46 common synthetic lethal candidate genes, 41 fell into one or more putative DNA damage response and repair pathways, including homologous recombination, double-stranded break repair, nuclear excision repair, and Fanconi anemia. These results were consistent with the roles of DNA damage detection and repair in cisplatin resistance. Overall, the CRISPR screen corroborated mechanisms of treatment resistance, yet nearly all of the hits were in pathways that remain a challenge to therapeutically target.

### Example 3

### NPEPPS is a Novel Determinant of Response to Cisplatin

As a way to prioritize the 46 common synthetic lethal genes, RNA sequencing was performed on all of the cell lines used in the examples described above grown in regular media conditions. In addition, and to gain further insight in the link between gene function and synthetic lethality, mass spectrometry-based proteomic profiling was performed on cell lysates of all cell lines, again in regular media conditions (FIG. 1A). These different -omic data types were used to identify and characterize genes associated with treatment response to either gemcitabine, cisplatin, or the gemcitabine plus cisplatin combination.

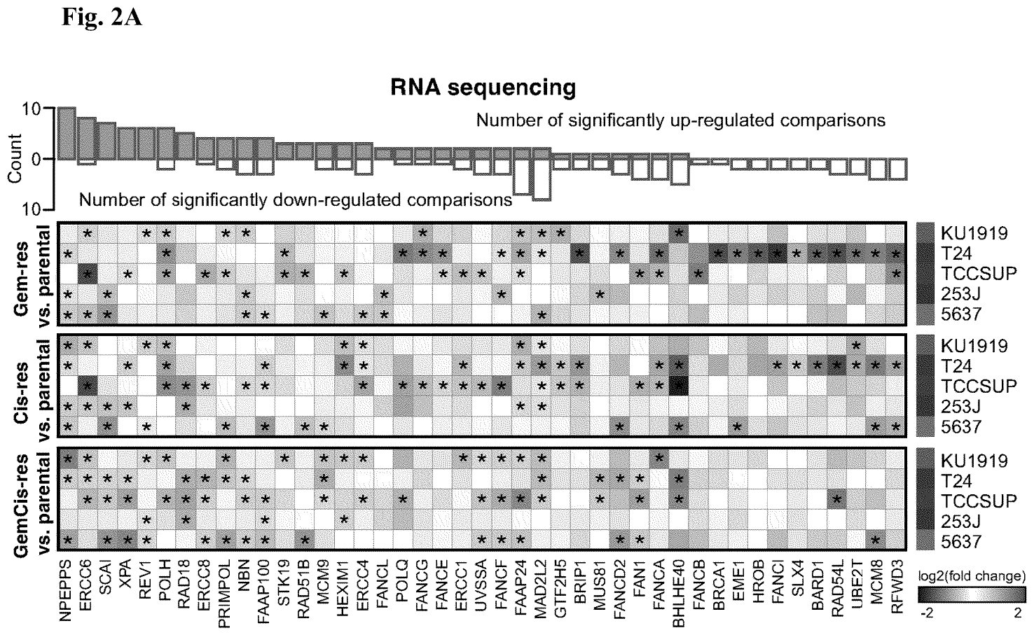

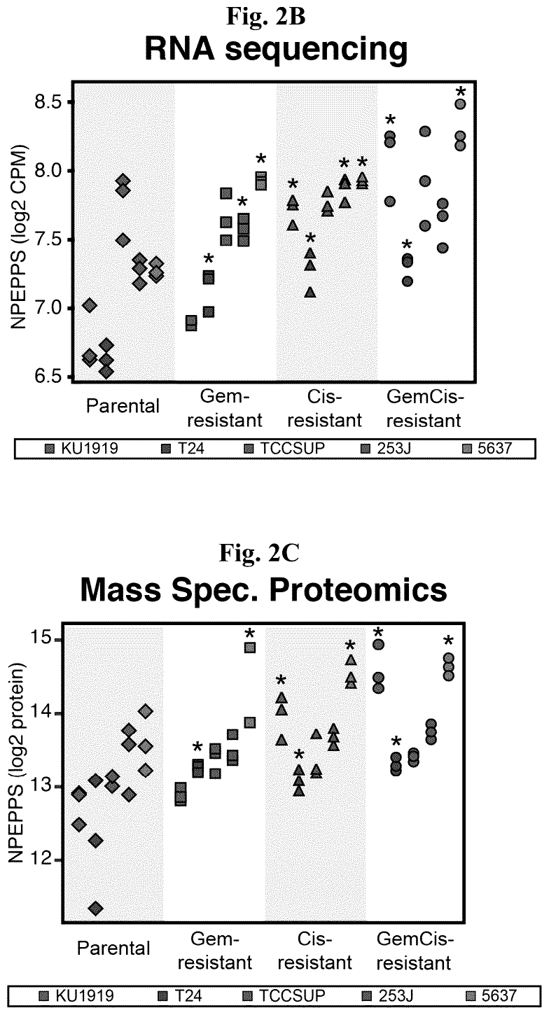

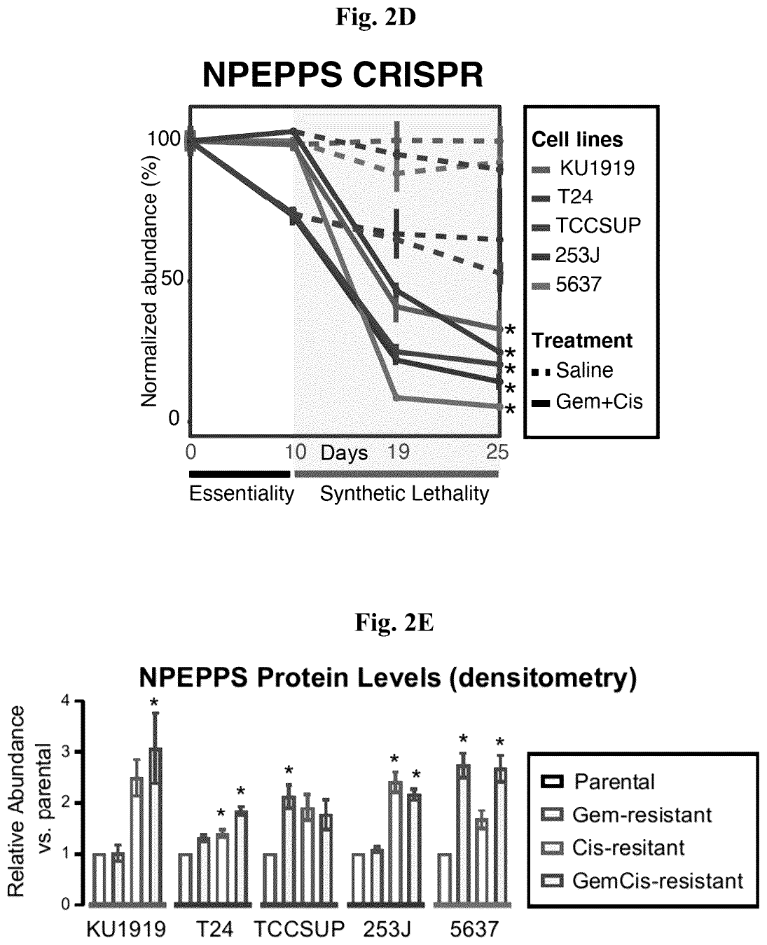

As the 46 synthetic lethal genes were identified as being significant across all 5 cell lines, gene and protein expression was similarly analyzed for each resistant derivative (Gem-, Cis-, and GemCis-) compared to the parental lines, while treating the cell type as a covariate in the statistical model. Data showed 1557 significantly upregulated genes across the Gem-resistant lines, 1897 in the Cis-resistant lines, and 1530 in the GemCis-resistant lines (moderated t-test, FDR<0.05). The proteomics data revealed 15 significantly upregulated proteins across the Gem-resistant cell lines (RRM1, FLNC, RHOG, MYH9, SVIL, DDX58, IVL, CNDP2, HARS1, CTSC, CRIP2, CHID1, EPS8L2, SLC9A3R1, CDC73), 1 in the Cis-resistant cell lines (EHD1), and 11 in the GemCis-resistant cell lines (RRM1, NPEPPS, GMDS, LAP3, XP076, SERPINB6, CORO7, PDLIM1, AK2, CNDP2, PSMD10) (moderated t-test, FDR<0.25). Given the lower number of significant proteins, the overlap between the CRISPR screen results and the transcriptome of the resistant cell lines was first investigated. There were few genes that were significantly and consistently up-regulated across the resistant derivatives and found to be in the list of 46 synthetic lethal genes (FIG. 2A), but the most significantly and consistently upregulated genes were involved in DNA damage response and repair mechanisms, including ERCC6, XPA, REV1, POLH, ERRC8, PRIMPOL, NBN, and members of the Fanconi Anemia pathway.

Puromycin sensitive aminopeptidase, NPEPPS, was identified as being the most consistently upregulated gene (FIGS. 2B and 2C) and one of the top synthetic lethal hits (FIG. 2D). Next, protein upregulation was determined through immunoblotting for NPEPPS across all cell lines and their derivatives. It was found that NPEPPS was consistently upregulated in the Cis-resistant and GemCis-resistant lines, with the Gem-resistant lines showing variable upregulation (FIG. 2E). The abundance of evidence supported NPEPPS as a gene that was commonly synthetic lethal and consistently upregulated across all of the resistance derivative cell lines.

In support of the novel role of NPEPPS in mediating cisplatin resistance, it was found that NPEPPS was specifically depleted in response to cisplatin, but not gemcitabine in an independent whole genome CRISPR screen that tested 27 genotoxic agents. This result also strongly supported the robustness of our findings, since this study used different CRISPR libraries (TKOv2 and TKOv3) and cell line (retinal pigment epithelium-1, RPE1). This study also supported the overall results of our screen, showing Fanconi Anemia and nucleotide excision repair related pathways as being highly significantly associated with cisplatin sensitivity. Nearly all of the 46 hits were significant and associated with cisplatin, but not gemcitabine.

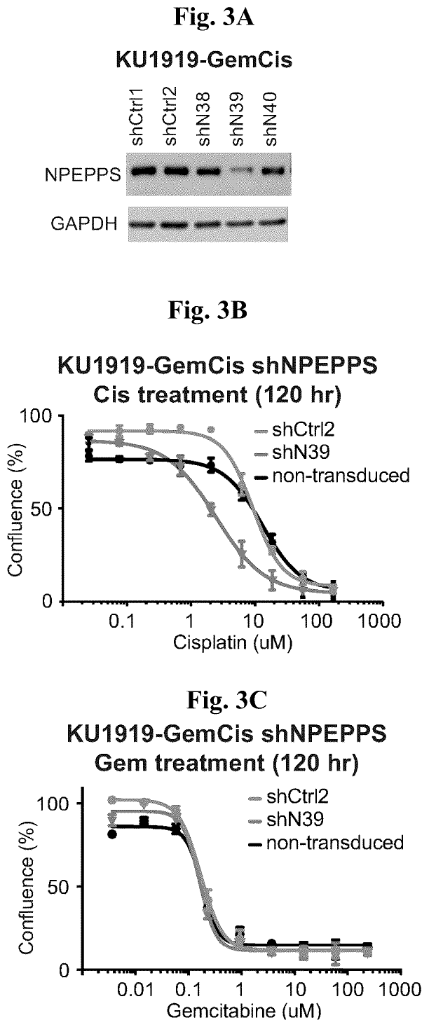

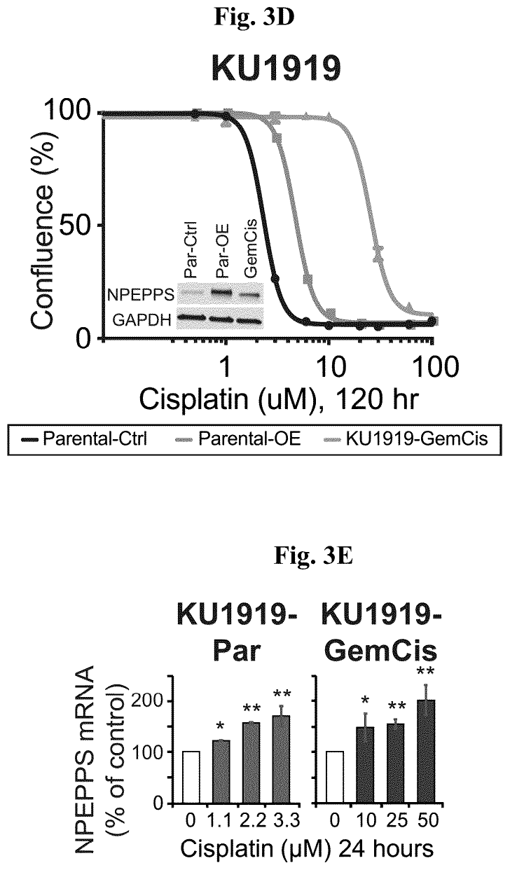

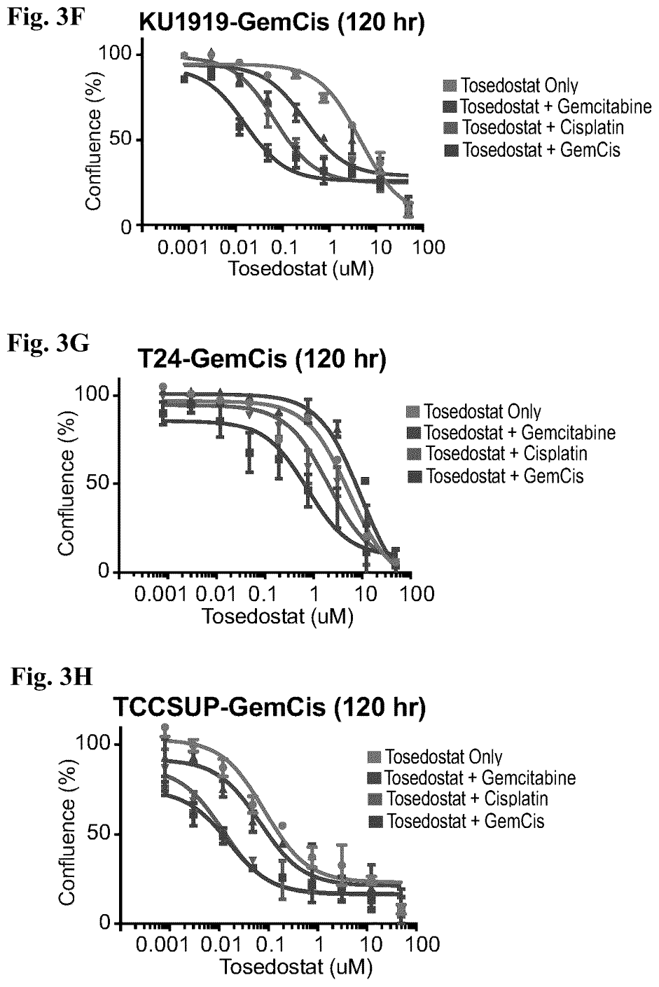

To validate that NPEPPS suppression enhanced sensitivity to gemcitabine plus cisplatin treatment in GemCis-resistant bladder cancer cells, and potentially showed a preference for affecting cisplatin sensitivity, stable NPEPPS shRNA knockdowns were generated in the KU1919-GemCis cell line (FIG. 3A). The KU1919-GemCis line was selected because it had the strongest combination of a synthetic lethal result and gene/protein upregulation (FIGS. 2A-2F). The impact of NPEPPS to cisplatin and gemcitabine sensitivity was tested separately and found that NPEPPS knockdown preferentially enhanced response to cisplatin in the combination, but not gemcitabine alone (FIGS. 3B and 3C). To further investigate, data showed the same result used siRNA targeting NPEPPS in the KU1919-GemCis cell line. Findings herein were further confirmed using siRNA in the KU1919-GemCis cell line and shRNA and/or siRNA in T24-GemCis and 253J-GemCis cells. Overexpression of NPEPPS in KU1919 parental lines led to increased treatment resistance to cisplatin (FIG. 3D), but not gemcitabine (data not shown). In addition, NPEPPS mRNA increased with cisplatin treatment in both KU1919-Par and KU1919-GemCis cells after 24 hours of treatment (FIG. 3E). These results established the role of NPEPPS in mediating sensitivity to gemcitabine plus cisplatin treatment and that this was primarily driven by its effect on mediating resistance to cisplatin in the gemcitabine plus cisplatin combination.

One feature of NPEPPS is the availability of agents that inhibit its activity. Tosedostat, an orally available M1 aminopeptidase prodrug has antileukemic activity and is readily available for use. Tosedostat is converted intracellularly into its active form (CHR-79888), which accumulates in cells because it is poorly membrane-permeable and inhibits the M1 aminopeptidase family. Tosedostat is antiproliferative, proapoptotic, and has antiangiogenic effects. The response of the KU1919, T24, and TCCSUP GemCis-resistant cells to increasing doses of tosedostat with gemcitabine, cisplatin, and gemcitabine plus cisplatin was measured. (FIGS. 3F-3H). Consistent with the genetic inhibition of NPEPPS shown herein, gemcitabine treatment showed little to no effect with tosedostat, with the strongest combined effect seen with cisplatin and gemcitabine plus cisplatin treatment. These results illustrate that GemCis-resistant bladder cancer cells can be re-sensitized to cisplatin-based treatments by genetic and pharmacologic inhibition of NPEPPS by inhibition of expression, for example.

### Example 4

Volume Regulated Anion Channels and their Impact Chemoresponse in Bladder Cancer Cells

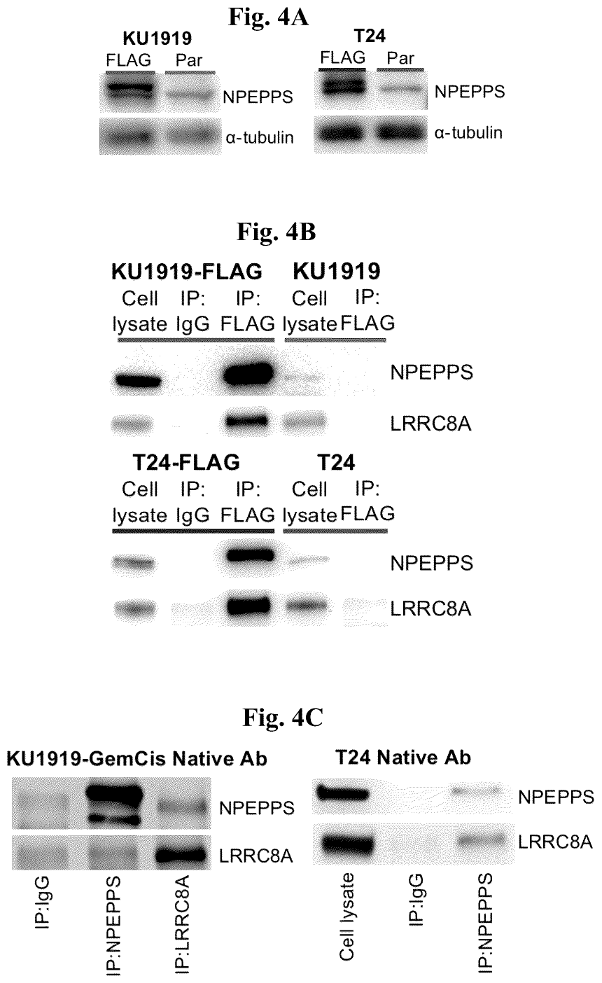

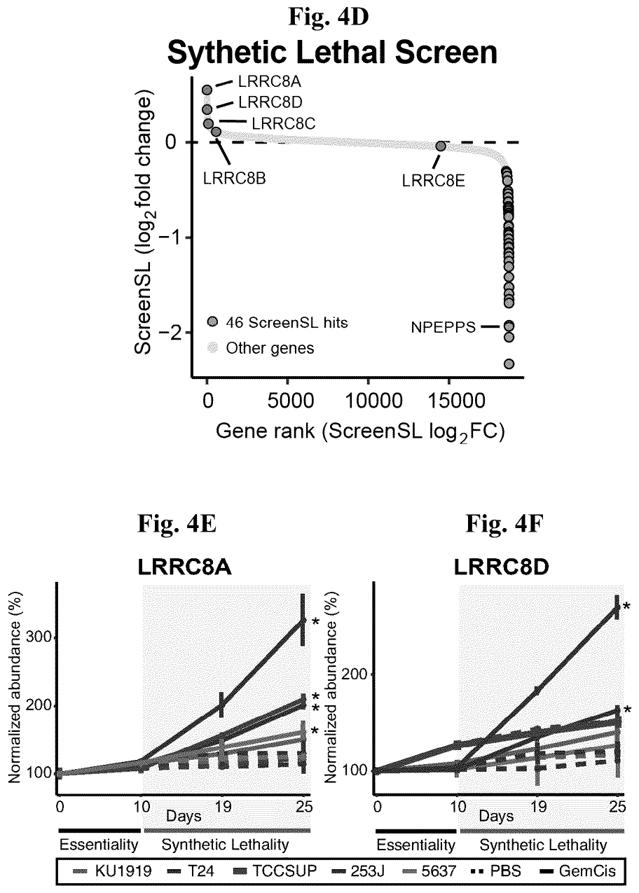

NPEPPS is one of 13, M1 aminopeptidase, which is a zinc dependent metallopeptidase, and as such cleaves the amino acids from the N-terminus of polypeptides. NPEPPS has been shown to be involved in a range of activities, including cell maintenance, growth, development and defense. However, NPEPPS role in chemotherapeutic response is unclear. NPEPPS protein interaction partners were investigated herein in the BioPlex interactome, a database that has collected affinity-purification mass spectrometry measurements of systematically over-expressed, tagged proteins. Among the small number of proteins that were observed to interact with NPEPPS, all five were members of the volume regulated anion channel (VRAC), LRRC8A-E. None of the other 12, M1 aminopeptidases were found in complex with any VRAC members in the BioPlex interactome and none of the other 12, M1 aminopeptidases were found to be synthetic lethal in the CRISPR screens performed herein (data not shown). To examine if the NPEPPS-VRAC interaction was present in bladder cancer cell lines, FLAG-tagged NPEPPS overexpressing KU1919 and T24 cells were generated (FIG. 4A). Cells were harvested and subjected to immunoprecipitation against FLAG followed by immunoblotting against NPEPPS and LRRC8A, the obligate channel member. LRRC8A reliably co-immunoprecipitated with NPEPPS in both cell lines (FIG. 4B). Additionally, using antibodies targeting the native protein, NPEPPS was pulled down and found LRRC8A, and complementary pulled down LRRC8A and found NPEPPS in the KU1919-GemCis cells (FIG. 4C). To verify this relationship in a separate parental cell line, NPEPPS was pulled down and found LRRC8A in T24 parental cells (FIG. 4C).

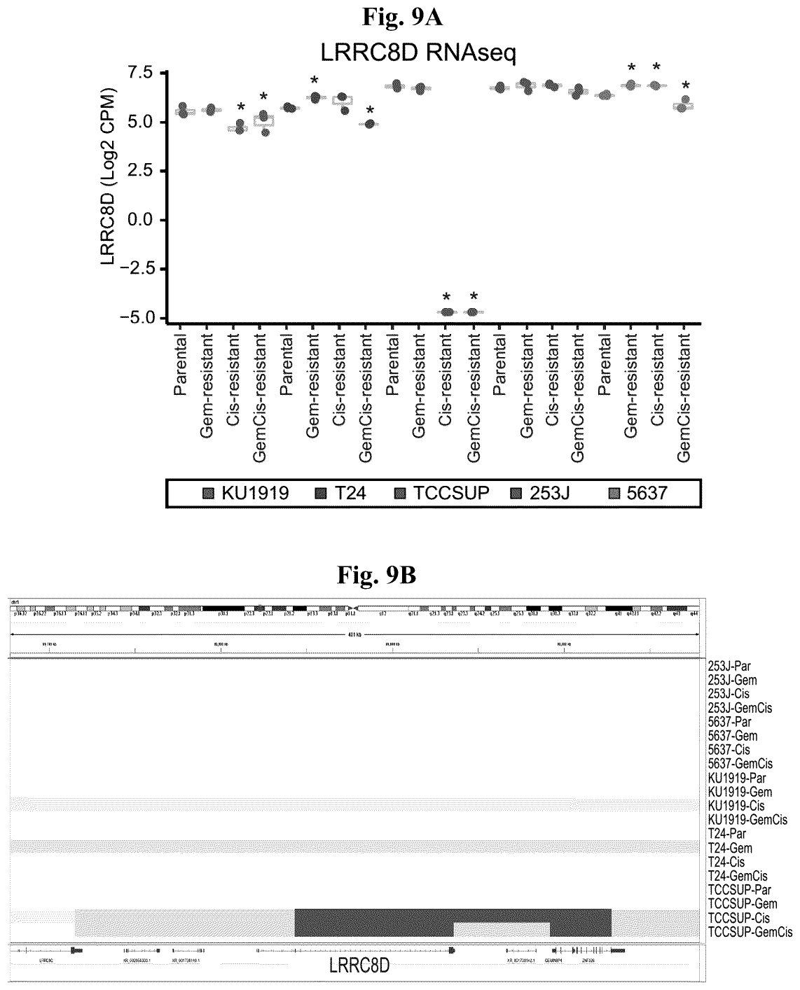

VRACs directly respond to osmotic stress by trafficking a wide range of organic osmolytes in and out of the cell to regulate cell volume. Importantly, channel subunits, LRRC8A and LRRC8D, have been previously identified in unbiased screens as mediators of platinum drug resistance, with it being reported that 50-70% of intracellular cisplatin is transported through these channels. Data herein demonstrated that NPEPPS negatively regulated of VRAC activity. As such, it was observed that NPEPPS blocks activity of the VRAC channel, which imports cisplatin, thus blocking cisplatin's ability to cause DNA damage and kill cells. By preventing NPEPPS from blocking VRAC activity, more cisplatin can enter into the cells and, in turn, kill the cells. LRRC8D gene expression in the TCCSUP-Cis and TCCSUP-GemCis cells was completely lost as a consequence of a deep deletion at the LRRC8D locus (FIGS. 9A-9B). Thus, further analysis herein was focused on the LRRC8A and LRRC8D subunits.

In another exemplary method, based on the associations between NPEPPS, LRRC8A/D, and cisplatin response, the CRISPR screens and RNAseq data herein were revisited to determine if loss of LRRC8A/D impacted acquired cisplatin resistance. Mass spectrometry proteomics did not reliably detect VRAC member peptides. Strikingly, LRRC8A was the 1 and LRRC8D was the 11′ ranked gene that when lost provided a growth advantage in gemcitabine plus cisplatin treatment across all cell lines (FIG. 4D). Opposite to NPEPPS, LRRC8A and LRRC8D loss in the CRISPR screen provided a significant growth advantage to cells treated with gemcitabine plus cisplatin (FIGS. 4E-4F). LRRC8A and/or LRRC8D gene expression was reduced in the Cis- or GemCis-resistant cell lines (data not shown). LRRC8D gene expression in the TCCSUP-Cis and TCCSUP-GemCis cells was completely lost (FIG. 4D). It was found that in these cell lines, there is a deep deletion at the LRRC8D locus (FIGS. 9A-9B). These combined results supported LRRC8A and LRRC8D loss as a possible genetic driver of cisplatin resistance.

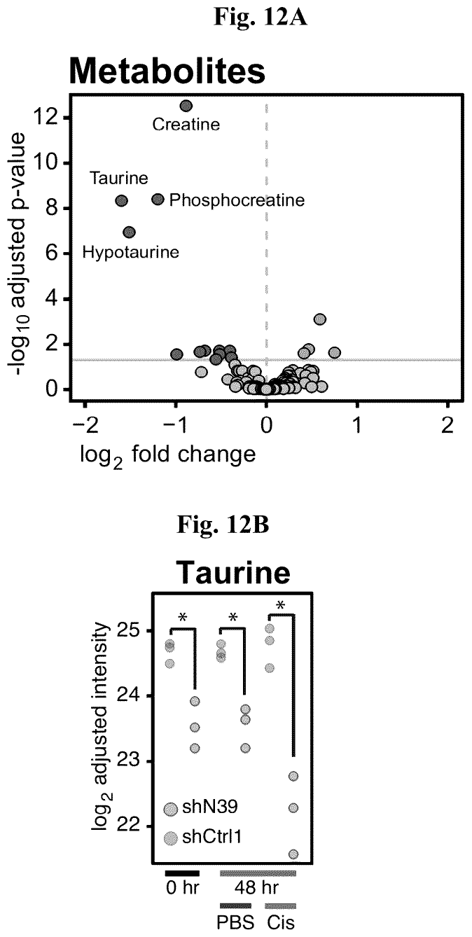

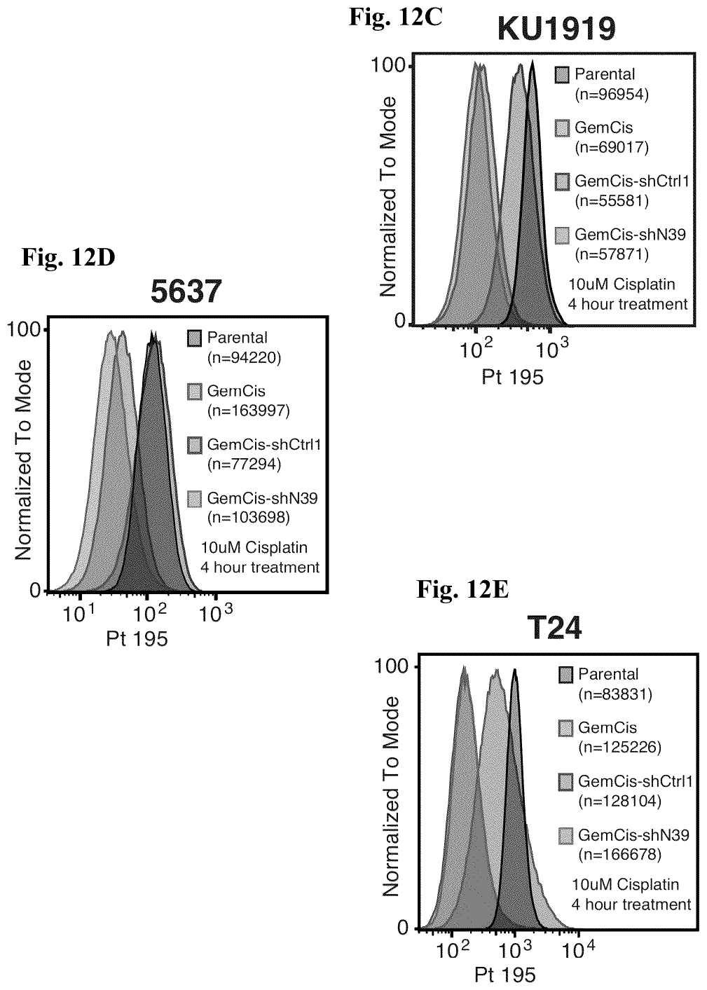

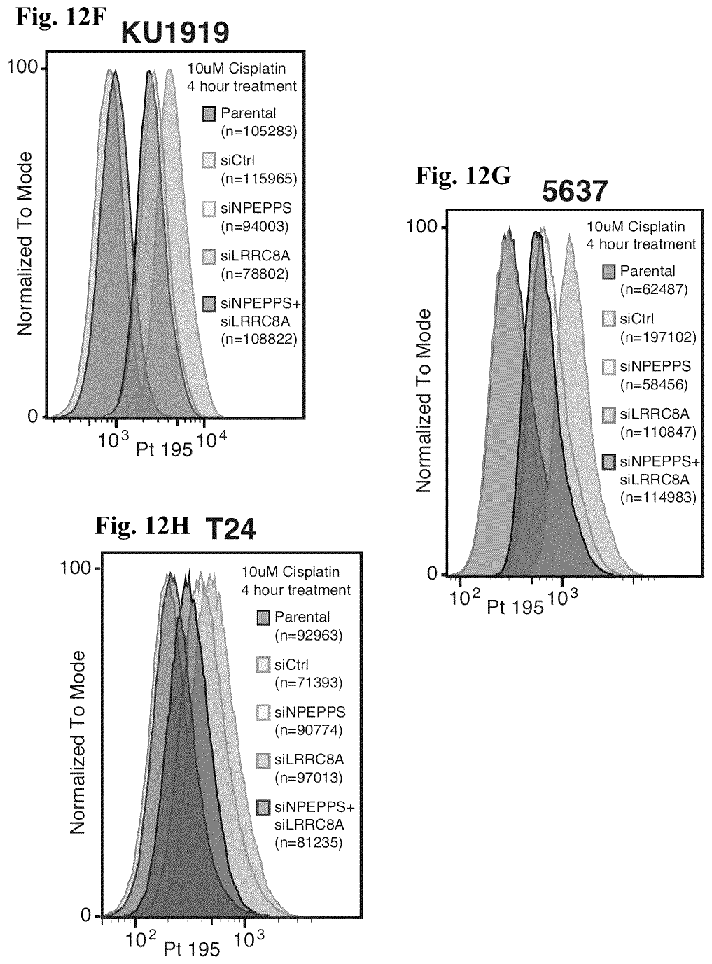

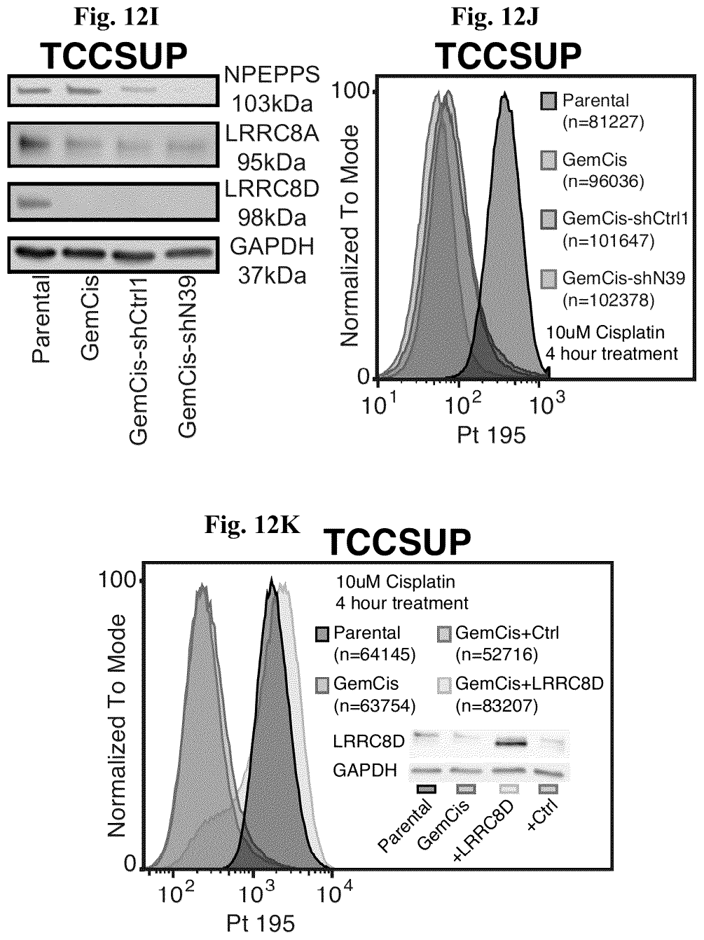

Given that VRACs transport cisplatin and carboplatin and finding NPEPPS in complex with VRAC subunits (FIGS. 4C-4D), NPEPPS may be a negative regulator of VRAC activity, consequently reducing import of intracellular cisplatin. Thus, the impact of NPEPPS on osmolytes known to be transported through VRACs was assessed. NPEPPS knockdown in KU1919-GemCis-shN39 cells resulted in significantly lower levels of intracellular taurine, hypotaurine, creatine, phosphocreatine, and several other amino acids (FIG. 12A) which are known to be exported via VRACs. In addition, intracellular levels of taurine were reduced even further when cells with knockdown of NPEPPS were also treated with 10 μM cisplatin (FIG. 12B). This suggested that cisplatin further stimulated channel activity when NPEPPS was decreased, which allowed for increased export of taurine, and increased cisplatin import.

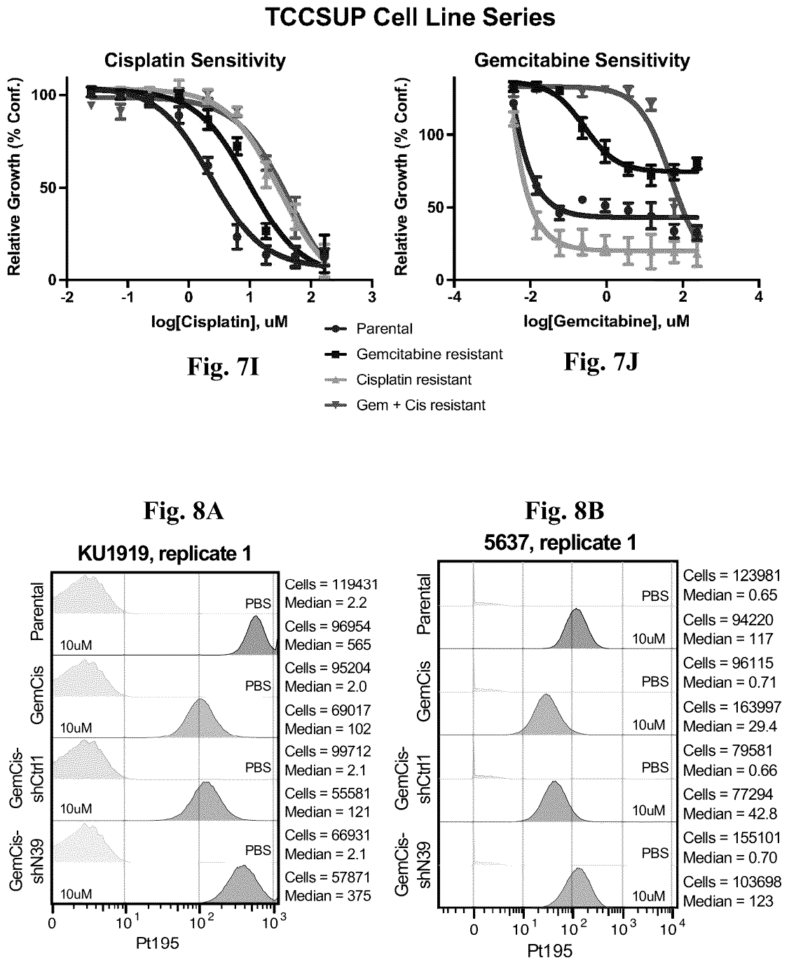

To evaluate NPEPPS impact on cisplatin import, intracellular cisplatin was directly measured using the metal ion detection capabilities of cytometry by time-of-flight, CyTOF. In brief, intracellular cisplatin was measured after 4 hours of treatment at 10 μM across KU1919, 5637, and T24 cell lines. Using KU1919 as the illustrative example, KU1919-GemCis cells (median Pt 195=102) showed decreased uptake of cisplatin compared to KU1919-Par cells (median Pt 195=565). Control knockdown had little effect (median Pt 195=121), but NPEPPS knockdown shifted the intracellular levels of cisplatin to that of parent lines (median Pt 195=375), suggesting that NPEPPS depletion allowed for increased import of cisplatin (FIG. 12C and FIG. 8A). These findings were repeated in the 5637 and T24 cell lines with highly similar results (FIGS. 12D-12E and FIG. 8B).

Furthermore, protein levels of LRRC8A and LRRC8D were measured after 48 hours of PBS or 10 μM cisplatin treatment in NPEPPS knockdown or nontargeting control KU1919-GemCis, 5637-GemCis, and T24-GemCis cells. Supporting the CyTOF results (FIGS. 12C-12E) and the result that taurine was exported at a higher rate upon cisplatin stimulation in the KU1919-GemCis-shN39 cells (FIG. 12B), NPEPPS knockdown increased DNA damage as measured by γH2AX foci; however, major changes in LRRC8A or LRRC8D expression in response to NPEPPS knockdown or cisplatin treatment were not observed (data not shown).

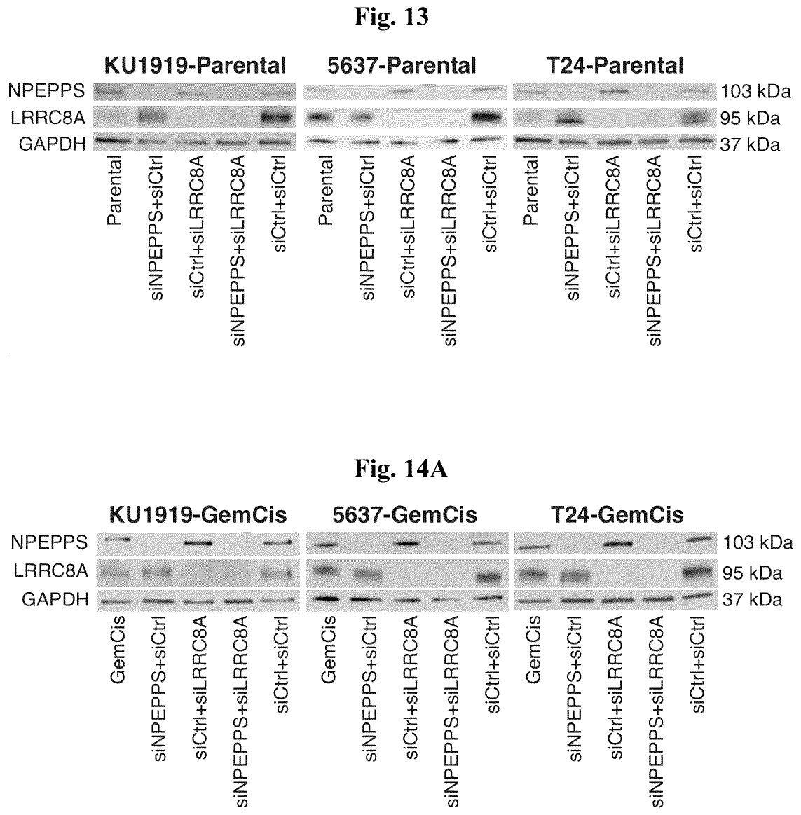

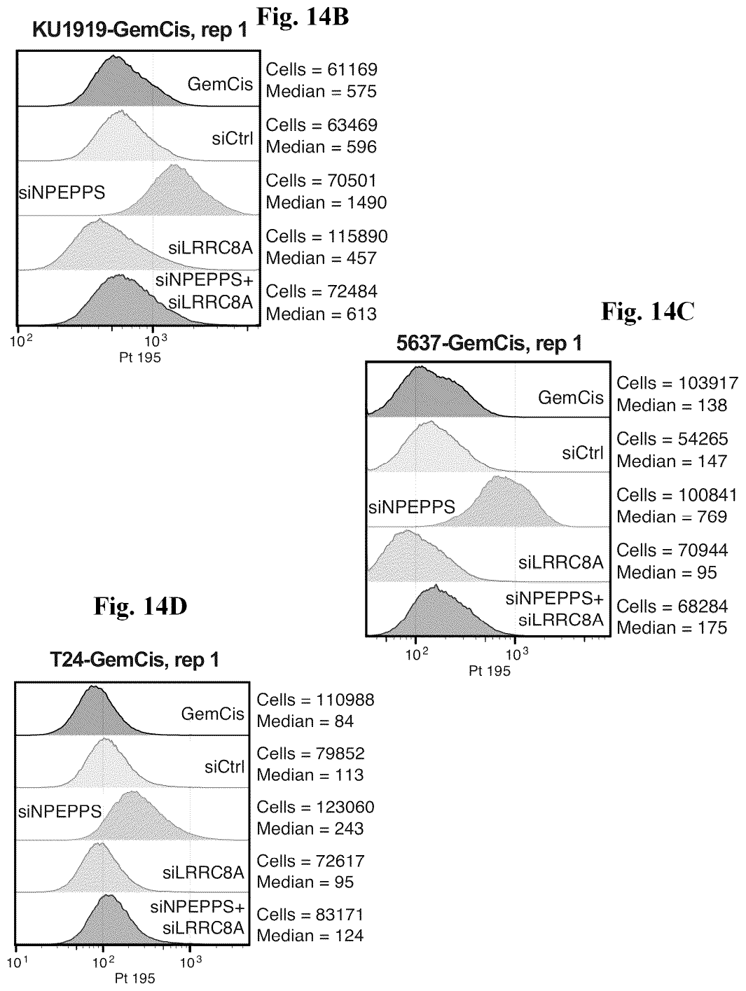

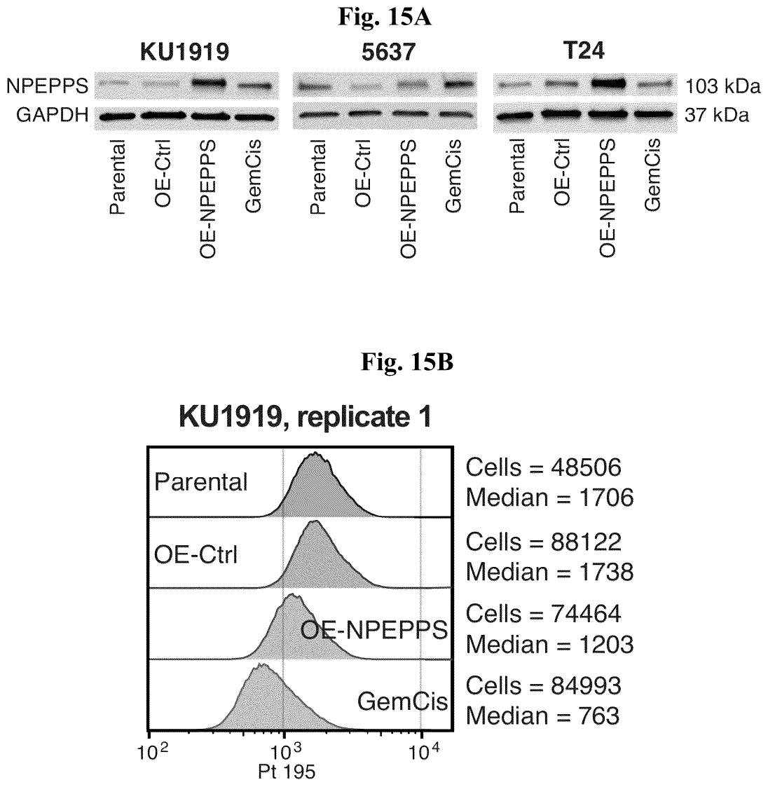

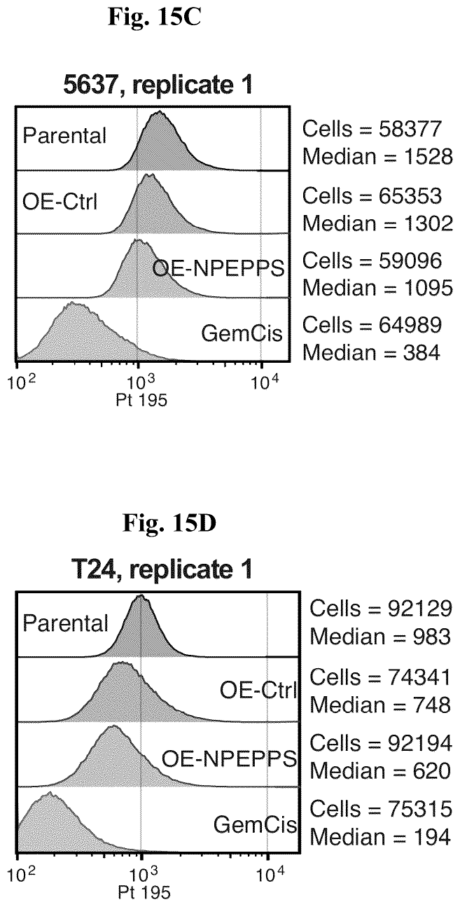

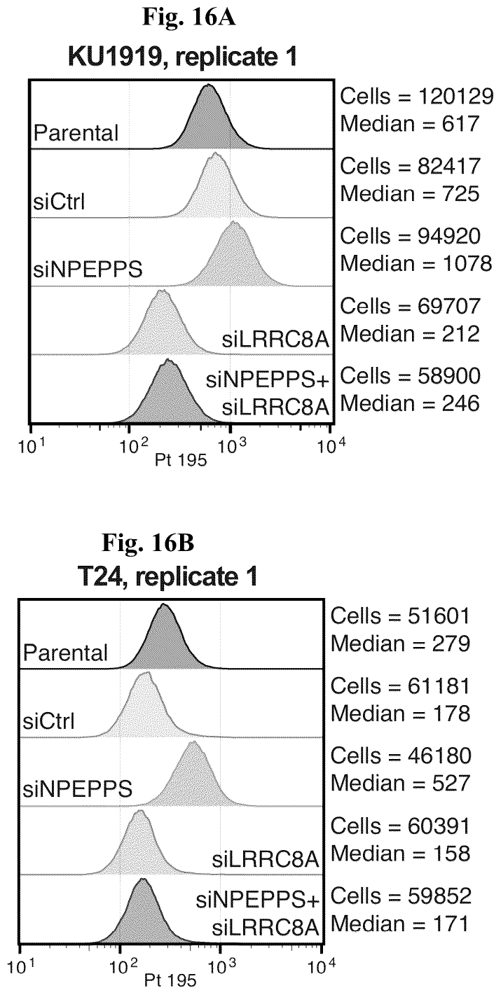

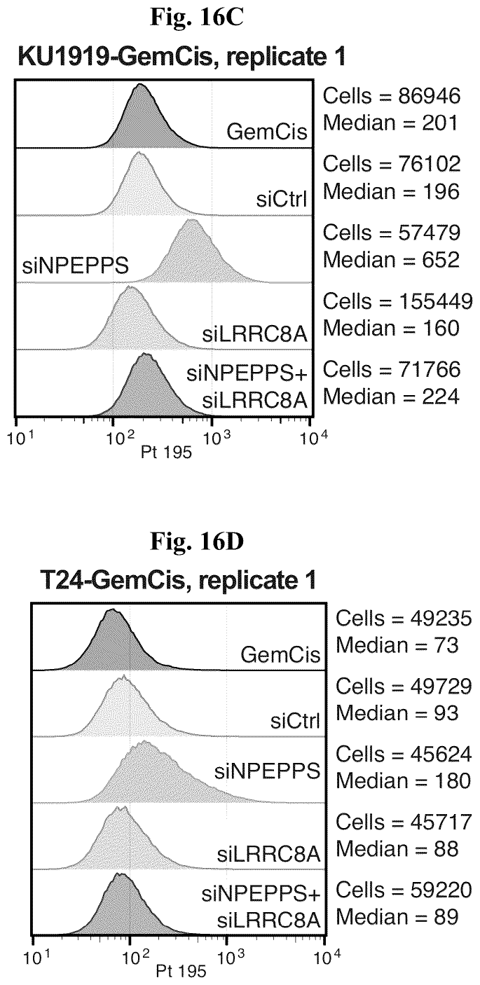

To determine the functional relationship between expression of NPEPPS and VRACs on intracellular cisplatin import, a series of siRNA experiments were performed targeting NPEPPS and/or LRRC8A (FIG. 13), the obligate subunit for normal VRAC function as mentioned above. The results shown in FIG. 13 support those shown in FIGS. 12F-12H. It was found that knockdown of NPEPPS in KU1919 parental cells increased import of cisplatin (KU1919 median Pt 195=1081; KU1919-siNPEPPS median Pt 195=1715), consistent with results from the GemCis-resistant cells (FIGS. 12C-12E). Knockdown of LRRC8A resulted in decreased intracellular cisplatin (median Pt 195=428), but knockdown of NPEPPS in combination with LRRC8A showed minimal additional effect (median Pt 195=498). These findings were reproduced in the 5637 and T24 cell lines (FIGS. 12F-12H). In addition, the same siRNA experiments were performed on the GemCis-resistant derivative cells (FIG. 14A). Depletion of LRRC8A did not result in additional resistance as these cells are already resistant, while NPEPPS knockdown alone resulted in increased intracellular cisplatin (FIGS. 14B-14D). Loss of NPEPPS had no effect when LRRC8A was also depleted (FIGS. 14B-14D). Overexpression of NPEPPS in KU1919, 5637, and T24 parental cells resulted in decreased intracellular cisplatin, suggesting that overexpression of NPEPPS is blocking cisplatin import (FIGS. 15A-15D). Finally, it was tested if carboplatin showed the same patterns of NPEPPS-mediated cisplatin import. Using KU1919 and T24 cells, the same patterns were found using carboplatin as were found with cisplatin (FIGS. 16A-16D).

To validate the role of LRRC8D in cisplatin import, intracellular cisplatin was measured in the TCCSUP cells. It was found that the parental cells imported much more cisplatin (median Pt 195=189) than the TCCSUP-GemCis cells (median Pt 195=22). In contrast to the KU1919, 5637, and T24 cells (FIGS. 12C-12E), NPEPPS knockdown had very little effect on the GemCis-resistant cells (median Pt 195=28) (FIGS. 12I-12J) given that the TCCSUP-GemCis cells have a focal deletion of LRRC8D (FIGS. 9A-9B). However, when LRRC8D was overexpressed, cisplatin import was restored to the levels of the parental cell line (FIG. 12K). Taken together, these data support a mechanism by which NPEPPS controls cisplatin import and subsequence cisplatin sensitivity through the VRACs.

### Example 5

### Genetic and Pharmacologic Inhibition of NPEPPS Enhances Chemotherapy Response In Vivo and Ex Vivo

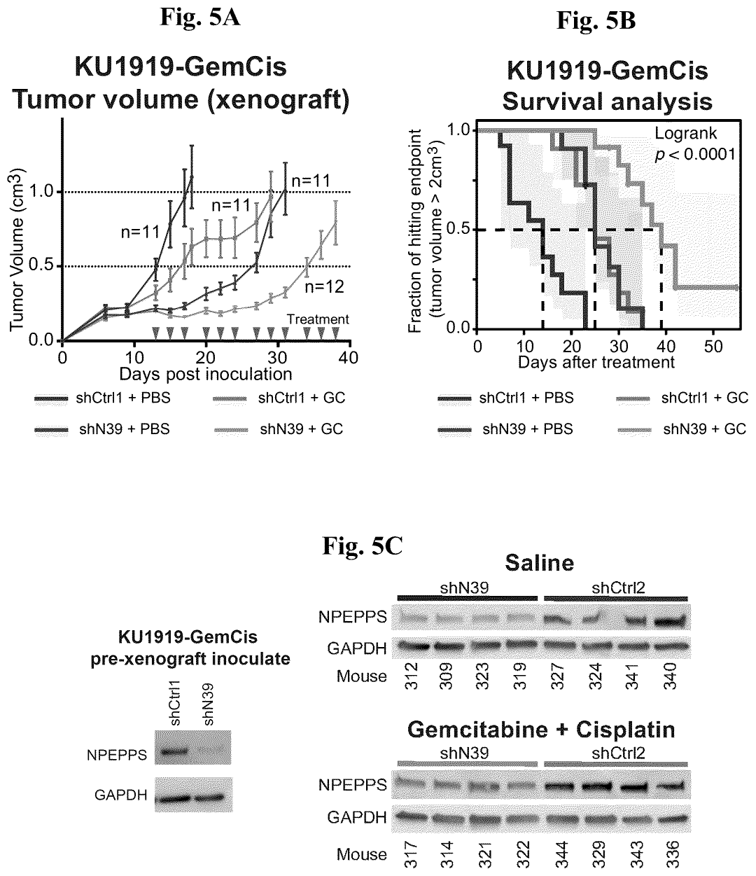

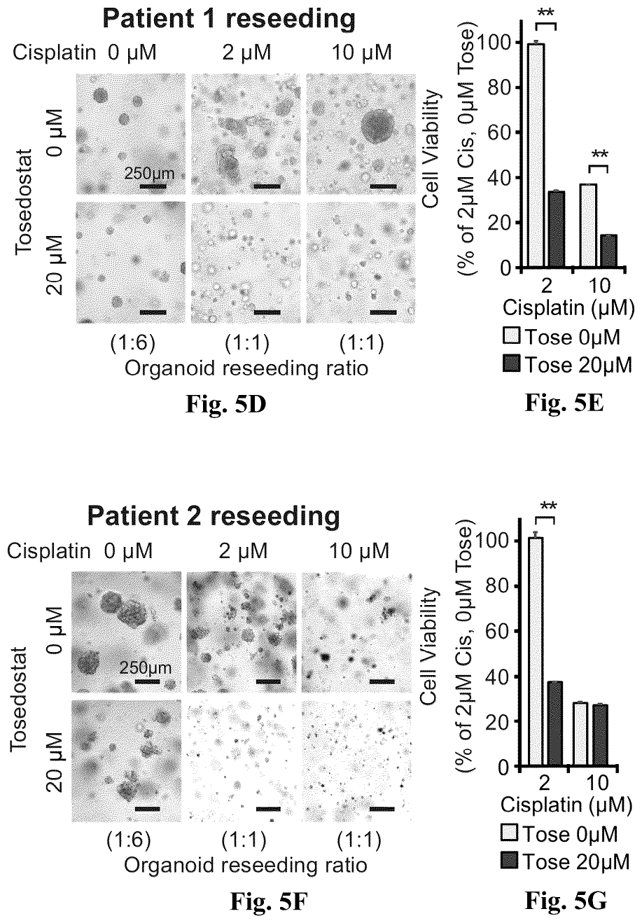

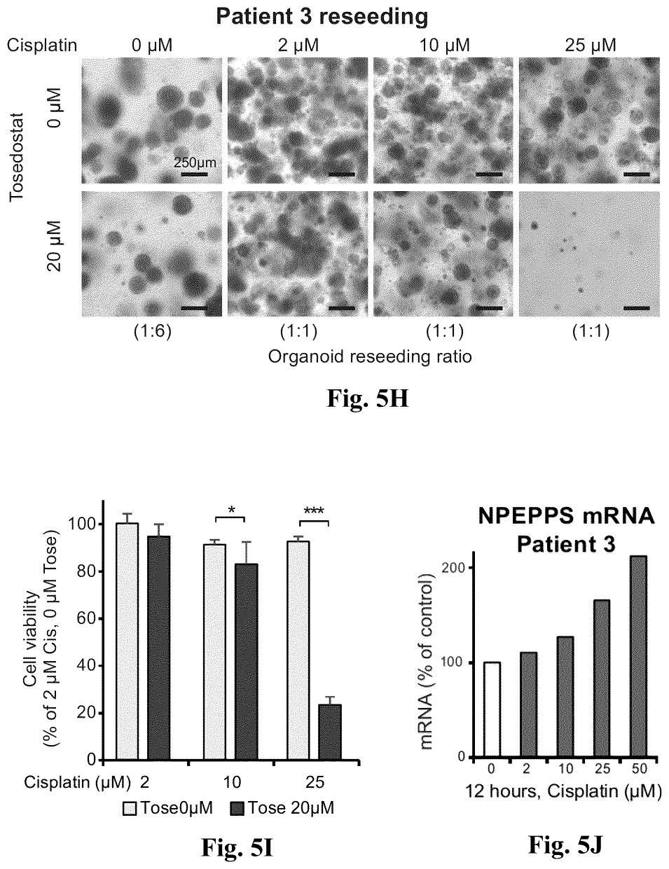

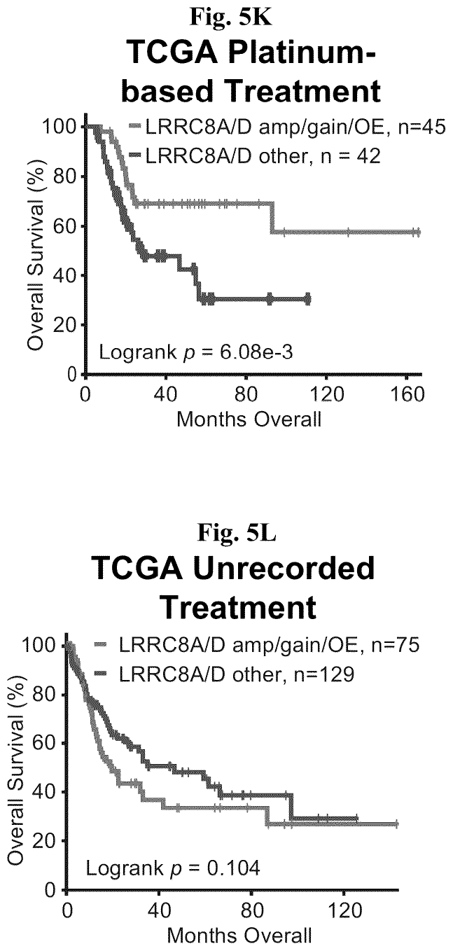

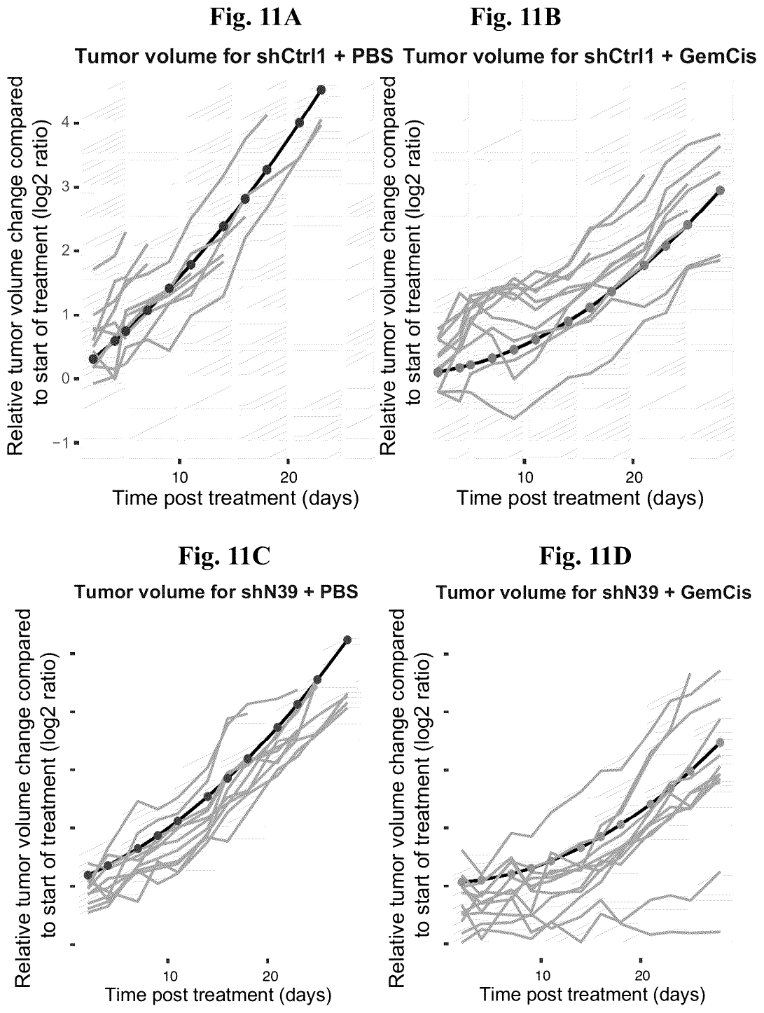

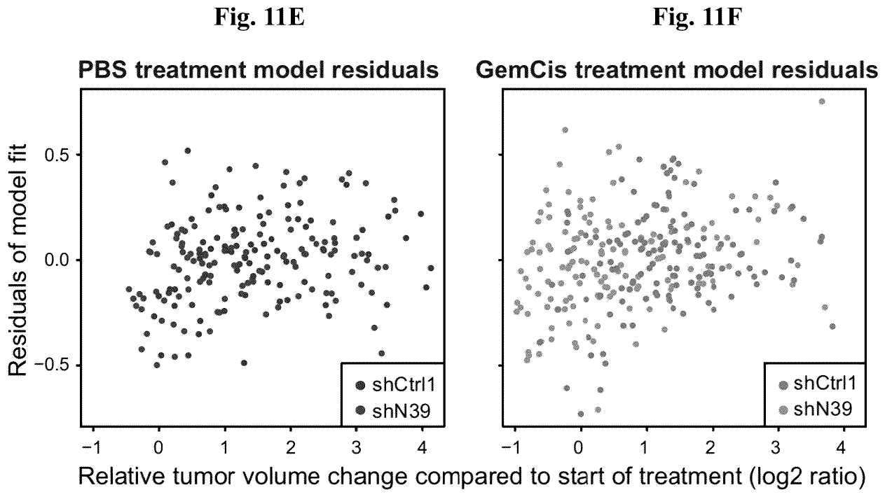

In another example, it was then tested whether NPEPPS depletion would re-sensitize tumor cells to gemcitabine plus cisplatin treatment in vivo. Subcutaneous xenografts were established using the KU1919-GemCis cells with either NPEPPS shRNA knockdown or non-targeting shRNA control. Mice were treated with saline (PBS) control or gemcitabine plus cisplatin three times weekly for four weeks and tumor volumes were monitored until they reached a predetermined endpoint of 2 cm³. NPEPPS knockdown alone and gemcitabine plus cisplatin treatment alone had significant impact on tumor growth compared to vehicle-treated, shRNA controls. The combination of NPEPPS knockdown and gemcitabine plus cisplatin treatment led to an even stronger and more significant impact on tumor growth (FIG. 5A). Tumor growth was analyzed using a linear mixed-effects model aimed at modeling tumor volume change in relation to pre-treatment baseline tumor volume across the four groups. Tumor growth was further analyzed using linear mixed-effects models aimed at capturing trends in tumor volume change in relation to pre-treatment baseline tumor volume across the four groups (FIGS. 11A-11F). According to this model, tumor growth inhibition by NPEPPS knockdown (p=0.00178), GemCis treatment (p=5.49e-07), or the combination of NPEPPS knockdown and gemcitabine plus cisplatin treatment (p=1.47e-08) were all consistent effects over the treatment period (FIGS. 5A-5B). NPEPPS knockdown was validated in the pre-xenograft inoculate cells and after tumors were removed from mice upon reaching the 2 cm³ endpoint (FIG. 5C). Survival analysis using tumor volume as the endpoint demonstrated that mice treated with gemcitabine plus cisplatin had a 14-day survival advantage. Similarly, knockdown of NPEPPS resulted in a 14-day survival advantage. Mice treated with gemcitabine plus cisplatin and with NPEPPS knockdown tumors had a 25-day survival advantage, a statistically significant result (Logrank test, p<0.0001) (FIG. 5B). The stability of NPEPPS knockdown for the duration of the experiment was confirmed using immunoblots from cells pre-xenograft and cells extracted from tumors at the endpoint of the study (FIG. 5C).

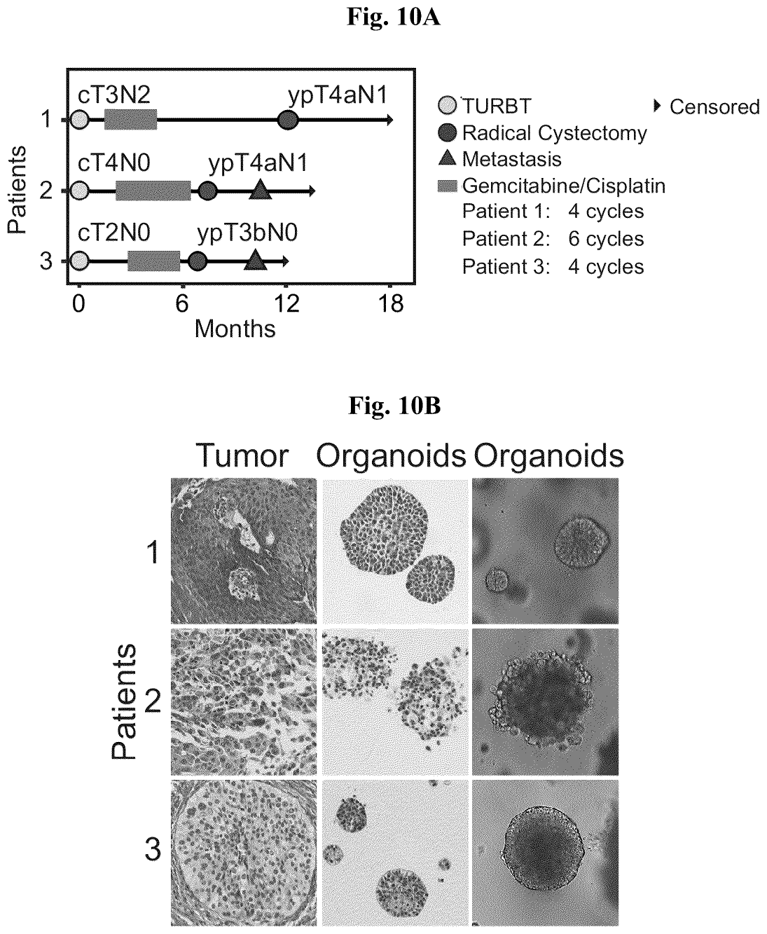

Given the demonstrated role of NPEPPS is at least cisplatin resistance, the impact of the drug combination of tosedostat and cisplatin was next evaluated on ex vivo human organoids. Three independent organoid models derived from patients that failed between 4 to 6 cycles of neoadjuvant gemcitabine plus cisplatin chemotherapy (FIG. 10A). The organoids mimicked the morphological and genomic characteristics of the tumor from which they were derived (FIG. 10B). These organoids were treated with increasing doses of cisplatin and 20 μM of tosedostat. Tosedostat alone had minor effect on organoid growth. In combination with 2 μM and 10 μM cisplatin, tosedostat significantly decreased organoid growth. At 25 μM, all organoids were killed by cisplatin alone; the maximum concentration of cisplatin in patients is 14 μM. Importantly, when organoids were reseeded in new growth conditions, which is analogous to a clonogenic assay, the tosedostat plus cisplatin combination demonstrated complete inhibition of growth, where the organoids were able to re-grow after being treated with 2 μM cisplatin alone. Organoids from patient 1 (FIGS. 5D and 5E) and patient 2 (FIGS. 5F and 5G) showed synergistic killing at 2 μM. Organoids from patient 3 (FIGS. 5H and 5I) were more treatment resistant with combination killing at 10 μM and had a synergistic effect at 25 μM. Notably, the maximum concentration of cisplatin in patients is 14 μM making our concentrations clinically relevant. Supporting results from FIG. 3E, organoids treated with cisplatin induced NPEPPS in a dose dependent manner (FIG. 5J). These results provided an additional and highly clinically relevant validation that tosedostat affects cellular response to cisplatin.

As demonstrated herein, NPEPPS was responsive to cisplatin treatment (FIG. 3E), suggesting that NPEPPS upregulation is an acquired mechanism of resistance to cisplatin-based chemotherapy. Therefore, pre-treatment NPEPPS levels would not necessarily be a biomarker of NAC response in bladder cancer. However, levels of LRRC8A and LRRC8D have been demonstrated to be predictive of cisplatin-based chemotherapy response in ovarian cancer. To evaluate the impact of genomic alterations of LRRC8A and LRRC8D in bladder cancer patients, TCGA MIBC genomic and overall survival data were analyzed. Patients treated with cisplatin, and also had an amplification, copy number gain, or increased expression of either LRRC8A or LRRC8D showed significantly improved overall survival (FIG. 5K), suggesting that these patients were responsive to treatment due to effective import of cisplatin into tumor cells. Conversely, in the group of patients that had unrecorded treatment information with the assumption that they did not receive cisplatin or carboplatin, the same LRRC8A and/or LRRC8D alterations were associated with significantly worse overall survival (FIG. 5L). Taken together, these findings supported VRAC subunits, specifically LRRC8A and LRRC8D, as pre-treatment biomarkers of cisplatin-based chemotherapy response.

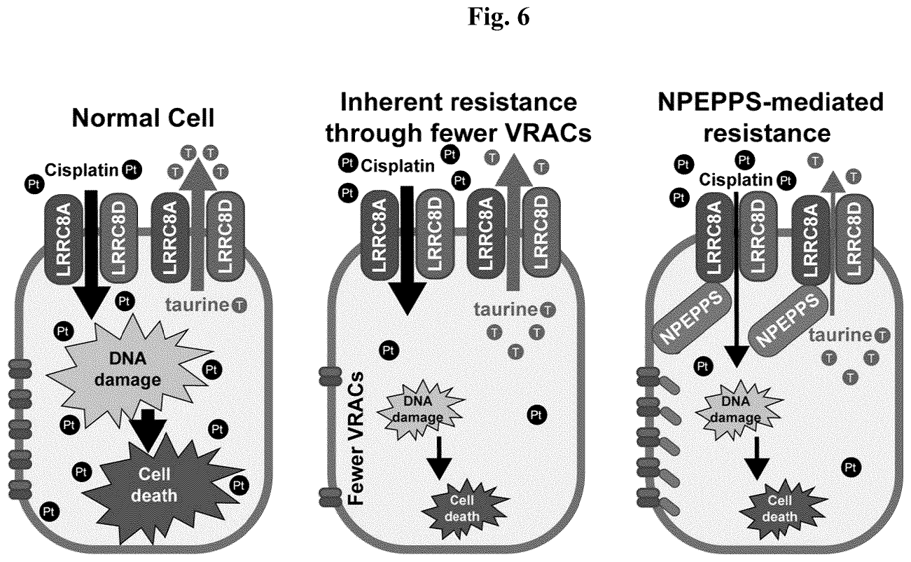

The data presented herein support a novel mechanism of NPEPPS-mediated chemotherapy resistance in bladder cancer, where NPEPPS interacted with VRACs to reduce the import of cisplatin, and thus providing resistance to the treatment. Illustrated in the schematic provided in FIG. 6, a typical cell was responsive to osmotic stress and able to import cisplatin, which caused DNA damage and eventual cell death. An inherent mechanism of resistance can simply be the number of VRACs in a tumor cells, where downregulation of VRAC subunits can lead to treatment resistance, such as was found in ovarian cancer, or the opposite effect seen in bladder cancer (data not shown). Data herein indicated that NPEPPS interacted with the VRACs, primarily LRRCA and LRRC8D, to inhibit the channels capacity, thus providing resistance to cisplatin and overall chemoresistance.

### Methods

The following methodologies were performed in the exemplary methods disclosed in herein.

Cell Culture. All human BCa cell lines used in Examples 1-5 herein were obtained from the Resistant Cancer Cell Line (RCCL) Collection and were grown in Iscove's Modified Dulbecco's Medium (IMDM) with 10% Fetal Bovine Serum (FBS). Cells were passaged every two to three days. Resistance to gemcitabine and cisplatin were confirmed at the reported resistance doses from the RCCL. Lentivirus production utilized 293FT cells (ThermoFisher), which were maintained in DMEM (high glucose) supplemented with 0.1 mM non-essential amino acids (NEAA), 6 mM L-glutamine, 1 mM sodium pyruvate, and 500 μg/mL geneticin (G418) with 10% FBS added. Cells were routinely monitored for mycoplasma and confirmed negative at multiple times during this study using MycoAlert (Lonza). All cells were grown at 37° C. with 5% CO₂ in a humidified incubator.

All molecular characterization efforts (RNA sequencing, whole exome sequencing, and mass spectrometric proteomics) were performed on cells from independent passages and in drug-free, complete media to identify stable molecular changes rather than treatment induced transient response. Cells were routinely passaged through drug-containing media at the resistant doses to confirm resistance was maintained and early passage cells were utilized whenever possible.

RNA sequencing. Sample preparation. All cell lines were grown for several passages in the absence of antibiotics, gemcitabine or cisplatin. Cell pellets were snap frozen from sub-confluent dishes from 3 separate passages (replicates) for each of the 20 cell lines sequenced (5 cell lines, each with 4 derivatives: parental, G-resistant, C-resistant, GC-resistant). RNA was extracted using the RNAeasy Plus Kit (Qiagen). Cells were lysed and passed through QIAShredder column (Qiagen) according to the manufacturer's protocol. gDNA elimination columns (Qiagen) were used to remove any residual gDNA from the purified RNA. RNA integrity was assessed on the High Sensitivity ScreenTape Assay on the Tape Station2200 (Agilent) and only samples with an RIN score of 8 or higher were used for sequencing. RNA library preparation was performed using the Universal Plus mRNA -Seq+UDI kit (Nugen) according to the manufacturer's specification. Each library was sequenced to a minimum of 40 million clusters or 80 million 150 bp paired-end reads on a NovaSeq 6000 instrument (Illumina) at the University of Colorado Cancer Center Genomics Shared Resource.

Data processing. Illumina adapters and the first 12 base pairs of each read were trimmed using BBDuk and reads <50 bp post trimming were discarded. Reads were aligned and quantified using STAR against the Ensembl human transcriptome (GRCh38.p12 genome (release 96)). Ensembl genes were mapped to HGNC gene symbols using HGNC and Ensembl BioMart. Gene counts were generated using the sum of counts for transcripts of the same gene. Lowly expressed genes were removed if mean raw count <1 or mean CPM (counts per million)<1 for the entire dataset. Reads were normalized to CPM using the edgeR R package. Differential expression was calculated using the voom function in the limma R package. In addition to two-group comparisons, single drug comparisons for all cell lines were generated with cell line as a covariate.

Pathway analysis. Gene set enrichment analysis was performed using the full list of genes ranked by fold change for the indicated comparison and the fgsea R package using gene sets from the Molecular Signatures Database (v7.0). General plots were generated with the ggplot2 and ggpubr R packages. Heatmaps were generated with the ComplexHeatmap R package following z-score transformation.

Proteomics. Sample preparation. All cell lines were grown for several passages in the absence of antibiotics, gemcitabine or cisplatin, then seeded at 100,000-200,000 cells per well and grown for 48 hours in IMDM+10% FBS. Approximately 48 hours after seeding cells the supernatant was aspirated and cells were washed 3 times with cold phosphate buffered saline (PBS). Cells were lysed in 100 μl of 8 M Urea, 50 mM Tris-HCl, pH 8.0. Lysates were transferred to pre-chilled 1.5 mL microcentrifuge tubes and centrifuged at 15000 RCF (relative centrifugal force) for 10 minutes to pellet. The supernatant was then transferred to a clean, pre-chilled tube and frozen. Lysate replicates were collected in triplicate from different passages. Cell pellets were lysed in 8 M Urea supplemented with 0.1% Rapigest MS compatible detergent. DNA was sheared using probe sonication, and protein concentration was estimated by BCA (Pierce, Thermo Scientific). A total of 30 μg protein per sample was aliquoted, and samples were diluted to <2 M urea concentration using 200 mM ammonium bicarbonate while also undergoing reduction with DTT (10 mM) and then alkylation with IAA (100 mM). The pH of diluted protein lysates was verified as between 7-8, and samples were digested with sequencing grade Trypsin/Lys-C enzyme (Promega) in the presence of 10% Acetonitrile for 16 hours at 37° C. Samples were acidified adding formic acid to 1%, and speed vac dehydration was used to evaporate acetonitrile. Peptides were desalted on C18 tips (Nest group) and dried to completion. Prior to MS, peptides were resuspended in 0.1% Formic Acid solution at 0.5 μg/L concentration with 1:40 synthetic iRT reference peptides (Biognosys).

Data acquisition. Peptides were analyzed by liquid chromatography coupled with mass spectrometry in data independent acquisition (DIA) mode. Briefly, 4 μL of digested sample were injected directly unto a 200 cm micro pillar array column (uPAC, Pharmafluidics) and separated over 120 minutes reversed phase gradient at 1200 nL/min and 60° C. The gradient of aqueous 0.1% formic acid (A) and 0.1% formic acid in acetonitrile (B) was implemented as follows: 2% B from 0 to 5 min, ramp to 4% B at 5.2 minutes, linear ramp to 28% B at 95 minutes, and ramp to 46% B at 120 minutes. After each analytical run, the column was flushed at 1200 n/min and 60° C. by injection of 50% methanol at 95% B for 25 minutes followed by a 10-minute ramp down to 2% B and a 5-minute equilibration to 2% B. The eluting peptides were electro sprayed through a 30 um bore stainless steel emitter (EvoSep) and analyzed on an Orbitrap Lumos using data independent acquisition (DIA) spanning the 400-1000 m/z range. Each DIA scan isolated a 4 m/z window with no overlap between windows, accumulated the ion current for a maximum of 54 seconds to a maximum AGC of 5E5, activated the selected ions by HCD set at 30% normalized collision energy, and analyzed the fragments in the 200-2000 m/z range using 30,000 resolution (m/z=200). After analysis of the full m/z range (150 DIA scans) a precursor scan was acquired over the 400-1000 m/z range at 60,000 resolution.

Peptide library generation. To construct a comprehensive peptide ion library for the analysis of human BCa several datasets, both internally generated and external publicly available data resources, were combined. First, previously published human bladder tumor proteomics experiment was utilized a by downloading raw files from the online data repository (ProteomeXchange, PXD010260) and searching them through an internal pipeline for data dependent acquisition MS analysis against the UniProt human reviewed canonical sequence database using internal peptides to perform retention time alignment. To this library, a sample specific library generated from DIA-Umpire extraction of pseudo-spectra was appended a from one full set of replicates from the experimental bladder tumor cell lines. A final, combined consensus spectrast library containing all peptide identifications made between the internal and external dataset was compiled and decoy sequences were appended.

Data analysis. Peptide identification was performed. Briefly, chromatograms and assigned peak groups were extracted using openSWATH against the custom BCa peptide assay library prepared as described above. False discovery rate for peptide identification was assigned using PyProphet and the TRIC algorithm was used to perform feature-alignment across multiple runs of different samples to maximize data completeness and reduce peak identification errors. Target peptides with a false discovery rate (FDR) of identification <1% in at least one dataset file, and up to 5% across all dataset files were included in the final results. SWATH2stats was used to convert data into the correct format for use with downstream software MSstats. Each individual data file was intensity normalized by dividing the raw fragment intensities to that files total MS2 signal. MSstats was used to convert fragment-level data into protein-level intensity estimates via the ‘quantData’ function, utilizing default parameters with the exception of data normalization, which was set to ‘FALSE’. For plotting purposes, protein intensities were VSN normalized, log-transformed, and replicate batch effects were removed using the removeBatchEffect function in the limma R package. The limma package was also used to calculate differential protein expression. Multiple hypothesis correction was performed using the Benjamin Hochberg method.

Whole exome sequencing. Sample preparation. All cell lines were grown for several passages in the absence of antibiotics, gemcitabine or cisplatin. Cell pellets were snap frozen from sub-confluent dishes for each of the 20 cell lines sequenced (5 cell lines, each with 4 derivatives: parental, Gem-resistant, Cis-resistant, GemCis-resistant). gDNA isolation was performed using the Puregene cell and tissue kit (Qiagen) with the addition of RNase A Solution (Qiagen) according to manufacturer's instructions. gDNA was quantified using a Qubit 4.0, then sheared using a Covaris S220 Sonicator to 200 bp. Libraries were constructed using the Sure Select All Exon v6 library kit (Agilent) following the XT library preparation workflow. Completed libraries were run on the 4200 Tape Station (Agilent) using D1000 screen tape. Libraries were quantitated using the Qubit, diluted to 4 nM prior to verification of cluster efficiency using qPCR, then sequenced on the NovaSeq 6000 instrument (Illumina) (150 bp, paired-end). Mean insert size across all cell lines was 177.8 bp and mean coverage was 193.7× with >96.8% at >30×.

Dataprocessing. The analysis pipeline was developed using Nextflow. For the raw fastq files, Fastqc was used to assess overall quality. For computational efficiency, raw sequence reads were partitioned using BBMap (partition.sh) into 40 partitions. They then were aligned to the GRCh38 reference genome (including decoy sequences from the GATK resource bundle) using the BWA-MEM short read aligner, and merged back into single BAM files using Samtools. The resulting BAM files were de-duplicated using Samblaster, and sorted using Samtools. These duplicate-marked barns were further passed through the GATK Base Quality Score Recalibration in order to detect systematic errors made by the sequencing machine when it estimates the accuracy of base calls. The dbSNP (version 146), the 1000 Genome Project Phase 1, and the Mills and 1000 G gold standard sets were used as databases of known polymorphic sites to exclude regions around known polymorphisms from analysis. After alignment, Samtools, Qualimap, and Picard tools were run to acquire various metrics to ensure there were no major anomalies in the aligned data.

Whole exome sequencing variant calling. Mutect2 from the GATK toolkit was used for SNVs and short indels. Mutect2 was designed to call somatic variants and makes no assumptions about the ploidy of samples. It was run in tumor-only mode to maximize the sensitivity albeit at the risk of high false positives. Tumor-only mode was used to call variants for each cell line separately. Mutect2 workflow was a two steps process. In the first step, it operated in high sensitivity mode to generate intermediate callsets that were further subjected to filtering to generate the final variant calls. Annotation of variants was performed using Annovar with the following databases: refGene, cytoBand, exac03, avsnp150, clinvar_20190305, gnomad211_exome, dbnsfp35c, cosmic90. Intergenic variants were removed along with variants that were identified at greater than 0.001% of the population according to ExAC or gnomAD or had a depth <20.

Copy number calling using GATK. Base quality score recalibrated bams were used as the input. The covered regions for the exome kit were converted into bins (defining the resolution of the analysis) for coverage collection. Read-counts, that form the basis of copy number variant detection, were collected for each bin. The read-counts then go through denoising, modelling segments, and calling the final copy ratios.

Cell line authentication. Variant calls from the Mutect2 pipeline were filtered for each cell line to identify high confidence variants according to the filtering criteria above. These high confidence variants were then compared to the variants reported for all cell lines in the DepMap for the Cancer Cell Line Encyclopedia and COSMIC as measured by the jaccard distance, the intersection of variants divided by the union of variants. Cells listed in CCLE or COSMIC were the rank ordered for each BCa cell line in this study according to the jaccard distance.

Metabolomics. Sample preparation. Cell lines were cultured for several passages in IMDM+10% FBS (IMDM10). Prior to experiment, cells were cultured in IMDM10 to ˜80% confluence and then dissociated. For dissociation, cells were washed once with room temperature PBS and then incubated with PBS+0.05% Trypsin-EDTA for 10-15 minutes. Cells were neutralized with IMDM10 and then fully dissociated by gentle pipetting. After dissociation, cells were counted by Trypan blue staining and then replated at 1×10⁶ cells. 24 hours after plating, cells were treated with either IMDM10 or IMDM10+10 μM cisplatin. Day 0 cell cultures were immediately processed for metabolomics analysis. To prepare cell pellets for metabolomics analysis, day 0 cells were dissociated and then centrifuged at 300 RCF for 10 minutes at 4° C. Cells were suspended in PBS, centrifuged a second time, and then resuspended in PBS and counted. Day 0 cells were centrifuged a third time, the supernatants were aspirated, and the dry cell pellets were snap frozen in liquid nitrogen and stored at −80° C. until metabolite extraction. 72 hours after plating, cells were processed for metabolomics analysis as described for the day 0 cell cultures.

Data generation and analysis. Metabolites from frozen cell pellets were extracted at 2×10⁶ cells/mL in ice cold 5:3:2 MeOH:acetonitrile:water. Extractions were carried out using vigorous vortexing for 30 minutes at 4° C. Supernatants were clarified by centrifugation (10 minutes, 18,000 g, 4° C.) and 10 μL analyzed using a Thermo Vanquish UHPLC coupled to a Thermo Q Exactive mass spectrometer. Global metabolomics analyses were performed using a 5 minutes C18 gradient in positive and negative ion modes (separate runs) with electrospray ionization. For all analyses, the MS scanned in MS¹ mode across the m/z range of 65 to 950. Peaks were annotated in conjunction with the KEGG database, integrated, and quality control performed using Maven. Data was variance stabilization normalized, log 2-transformed, and differential abundance calculations were done using limma with time and/or treatment as covariates in the linear model.

Cell Line Drug Treatments. Gemcitabine (Sigma) and cisplatin (Sigma) stocks were resuspended in 0.9% saline solution and tosedostat (Sigma and BOC Sciences) was resuspended in DMSO. All stocks solutions were stored protected from light and kept frozen until use. For cell culture dose response, cells were seeded in 96-well tissue culture plates with 500-2000 cells per well depending on growth rate and duration of experiment. Cells were seeded and allowed to attach overnight followed by replacing the media with fresh, pre-warmed media just prior to treatment. Drug dilutions were performed serially and using complete media (IMDM+10% FBS) and the associated drug treatments. Growth inhibition was measured using confluence estimates over time on the IncuCyte ZOOM (Essen Bioscience) over varying amounts of time depending on each experiment. Details for timing and replicates for each experiment are included in their respective figure legends.

Antibodies and Western Blotting. Whole cell lysates were prepared from cultured cells using RIPA lysis and extraction buffer (ThermoScientific). Lysates from xenograft tissues were prepared using tissue protein extraction reagent (T-PER) and glass tissue homogenizer. All lysates were prepared on ice and with the addition of Halt protease and phosphatase inhibitor cocktail and EDTA (ThermoFisher). Protein concentration of lysates were quantified with BCA protein assay (Pierce™, ThermoFisher). All lysates were prepared with 4× Licor Loading buffer with DTT added boiled for 10 minutes prior to gel loading. All western blots were run using PROTEAN TGX precast 4-15% or 4-20% gradient gels (Bio-Rad) and transferred to either 0.2 μm or 0.44 μm nitrocellulose membranes. Transfer was done for 1.5-2 hours in cold TrisGlycine buffer (Bio-Rad) with 20% methanol prior blocking for 1 hour at room temperature (20° C.±5° C.) in 5% BSA in TBS-T. Primary antibodies were diluted and incubated overnight at 4° C. on a rocker. Membranes were washed 3 or 4 times in fresh TBS-T prior a 1 hour room temperature incubation in an appropriate secondary antibody. Membranes were washed 3-4 times in TBS-T, developed with enhanced SuperSignal West Pico Plus or SuperSignal West Fempto (ThermoFisher) and imaged using Li-Cor Odyssey Fc instrument. Densitometry was performed using LiCor Image Studio software. Statistical comparisons using densitometry measurements were done using a one-way ANOVA with Tukey post hoc to control for the experiment-wise error rate.

Immunoprecipitation. Immunoprecipitation of human bladder cancer cell lines was carried out using Protein G Sepharose beads following manufacturer protocol (GE healthcare). Cells were lysed using Pierce IP lysis buffer containing 25 mM Tris HCL pH 7.4, 150 mM NaCl, 1% NP-40, 1 mM EDTA, 5% glycerol added with phosphatase and protease inhibitor mixture (Roche Applied Sciences). Sepharose beads slurry was washed three times with the lysis buffer by centrifuging at 3,000×g for 2 minutes at 4° C. Then conjugated anti-FLAG antibody was carried out by overnight incubation of the suspended Protein G Sepharose and anti-Flag monoclonal antibody (Sigma F1804) at 4° C. with continuous mixing. After three-time washing with lysis buffer, the mixture was incubated with the lysates at 4° C. overnight with gentle mixing on a suitable shaker. Next, the precipitated protein with the bead was washed three times and analyzed using the immunoblotting technique (i.e., Western blotting as described herein). Whole-cell lysate were used for input or positive control. Anti-FLAP pull down was performed for FLAG non-expressing bladder cancer cell line for negative control. NPEPPS and LRRC8A were probed using Rabbit polyclonal NPEPPS antibody (1:1000; Origene), Rabbit IgG polyclonal LRRC8A antibody (1:1000, LSBio) and Rabbit IgG polyclonal LRRC8D antibody (1:1000, SinoBiological).

Cisplatin induced NPEPPS mRNA expression. Total RNA was isolated from cells using Trizol (ThermoFisher) and/or phenol-chloroform based extraction methods. Residual DNA was digested with DNAse I (Life technologies). cDNA synthesis was performed using Superscript II Reverse Transcriptase kit (Life technologies) using random primers. RT-qPCR reactions were performed on a CFX Connect Real-Time PCR Detection System thermocycler (Bio-Rad) using TaqMan gene expression assays for NPEPPS and HMBS as a housekeeping gene (ThermoFisher) in combination with SensiFAST Probe No-ROX Kit (Bioline, Toronto, Canada). Expression data was calculated using 2^{-ΔΔCt}. All cell line experiments were performed in triplicate from independently grown cells. Comparisons at the indicated dose of cisplatin were made to the control treatment (0 μM cisplatin) using a t-test.

siRNA-mediated knockdown experiments. NPEPPS and non-targeting siRNA SMARTpools were resuspended in Dharmacon 5× siRNA Buffer. Transfections were performed using Lipofectamine RNAiMax (ThermoFisher) transfection reagent according to the manufacturer's specifications. Briefly, cells were grown to ˜60% confluence in 6-well plates prior to being transfected and allowed to incubate overnight (˜12-16 hours). The following day cells were trypsinized and replated into 96-well plates at 1000-2000 cells per well and allowed to attach overnight. Cells from the initial transfection were also replated into 6-well plates to collect protein and RNA to confirm knockdown. The following day, cells were treated using their previously established resistance doses of gemcitabine, cisplatin, or gemcitabine plus cisplatin, and their relative growth rates were measured on the IncuCyte ZOOM (Essen Bioscience) over time. For the CyTOF experiments, cells were grown in siRNA SMARTpools for 72 hours before beginning cisplatin treatment.

shRNA-mediated knockdown experiments. Lentiviral production and transduction were carried out as follows. In brief, plasmids from the RNAi Consortium (TRC) collection (TRC construct numbers TRCN0000073838, TRCN0000073839 and TRCN0000073840) were used for targeting NPEPPS were selected based on predicted knockdown efficiency; non-targeting controls used were SHC002 and SHC016. 2 μg of target shRNA construct and 2 μg of 3:1 ratio of psPAX2 (Addgene) and pMD2.G (Addgene) were transfected into HEK293FT cells using 2 μg of Polyethylenimine (Polysciences). Lentiviral particles containing media was filtered using 0.45 μm cellulose acetate syringe filter and used for transduction. Puromycin selection was performed at doses used for CRISPR library screening or in some cases, cells were re-selected with higher doses of puromycin (10 μg/mL), in order to ensure complete elimination of non-transduced cells. Selected cells were frozen at early passage and early passage cells were used for all experiments.

Intracellular cisplatin measurements using CyTOF. Cell lines were cultured for several passages in IMDM+10% FBS. Prior to experiment, cells were cultured in IMDM10 to be 50-80% confluence overnight and then treated the next day with varying concentrations of cisplatin or PBS as indicated and then dissociated after 4 hours of treatment. For dissociation, cells were washed twice with room temperature PBS and then incubated with PBS+0.05% Trypsin-EDTA for 10-15 minutes. Cells were neutralized with IMDM10 and then fully dissociated into single-cell suspension by gentle pipetting. After dissociation, cells were counted by Trypan blue staining and then placed in separate tubes at 3×10⁵ cells. Individual samples were then fixed, permeabilized, and labeled using unique barcodes using the Cell-ID 20-plex Pd Barcoding kit (Fluidigm) according to the manufacturer protocol. Barcoded samples were pooled across cell line condition and cisplatin concentration, incubated with Cell-ID Intercalator-Ir, mixed with equilibration beads and acquired on a Helios mass cytometer (Fluidigm). Post-acquisition data were normalized to equilibration beads and debarcoded, using the bead-normalization and single-cell-debarcoder packages from the Nolan Laboratory GitHub page. Relative cisplatin intensity (defined by ¹⁹⁵Platinum isotopic mass intensity) was analyzed among nucleated ¹⁹¹Iridium+¹⁹³Iridium+events defined by Boolean gating within FlowJo 10.7.1.

Whole Genome CRISPR Screening. Plasmid library expansion and quality control. Whole genome CRISPR Screening was performed using the Human CRISPR Knockout Pooled Library (Brunello)-1 vector system. Two distinct plasmid expansions were performed. The library distribution was assessed using next generation sequencing to determine the impact on overall library was modest following re-expansion. Library width was calculated by dividing the 10′ percentile of the library distribution by the 90′1 percentile using the log 2 average expression of all sgRNAs in the library and found to be 6.7 and 7.13 for batch 1 and 2 respectively.

Lentivirus Production and Titration. For the two plasmid batches, two distinct protocols for lentivirus production were utilized. The first batch was generated by using Polyethylenimine, linear (PEI; Polysciences) and was used for the T24-GemCis and TCCSUP-GemCis screens. The second used lipofectamine 3000 and was applied for the 253J-GemCis, KU1919-GemCis, and 5637-GemCis screens. For the first batch, 293FT cells were seeded at a density of 36,800 cells/cm² into a 4-layer CELLdisc (Greiner) using DMEM+10% FBS along with antibiotic and antimycotic solution. Transfection mix consisting 47.6 μg pMD2 G (Addgene), 95.2 μg of psPAX2 (Addgene), and 190.5 μg of Brunello Whole genome knockout library (Addgene) was mixed with 448 μl PEI (1 mg/mL) and 3 mL OptiMEM, vortexed for 30 seconds and allowed to incubate at room temperature for 20 minutes. Fresh media containing transfection mix were added to the CELLdisc using up to 270 mL of media. The next day media was changed for 280 mL fresh media followed by a 48-hour incubation. After this 48-hour incubation the viral supernatant was harvested and filtered through a cellulose acetate filter system (ThermoScientific) and frozen at −80° C.

In a second batch of virus production, lipofectamine 3000 was utilized instead of PEI, multilayer flasks were used, and cells were centrifuged to remove debris as opposed to filtering. Briefly, 293FT cells were plated in T225 flasks to be 80% confluent after 24 hours. Two hours before transfection, media was changed and 40 mL of fresh media was used per T225 flask. The lipofectamine 3000 protocol was followed according to manufacturer's instructions and scaled based on the volume of virus being prepared. For each T225 flask 2 mL OptiMEM was mixed with 40 μg Brunello whole genome library plasmid, 30 μg of psPAX2 and 20 μg of pMD2.G and 180 μl of P3000. This mix was added to a tube containing 2 mL OptiMEM and 128 μl Lipofectamine 3000, which was scaled according to the number of T225 flasks being prepared. Transfection mix was mixed thoroughly by pipetting up and down slowly, and allowed to incubate at room temperature for 15 minutes. Transfection mix was then added dropwise to the plates of 293FT cells with gentle swirling and incubated overnight (˜16 hours). The following morning, the media was changed and 60 mL of fresh media was added to each T225 flask. This was incubated overnight and replaced the following morning. This first lentiviral supernatant was stored at 4° C. to be pooled with a subsequent 48 hour collection. Upon collection, viral supernatants had 1 M HEPES added at 1%. Following the second virus collection, supernatants were pooled and centrifuged at 1250 rpm for 5 minutes to pellet debris. Lentivirus was stored in polypropylene tubes as polystyrene is known to bind lentivirus, and all tubes were flash frozen in liquid nitrogen and stored at −80° C. Despite the changes to the lentiviral production protocols, functional lentiviral titers were not improved using these changes to the methodology, but feel it is worth noting these changes in protocol to account for any possible variability associated with this change. Lentivirus was titered functionally.

DNA Isolation. Cell pellets of 2×10⁷ were snap frozen in liquid nitrogen in 1.5 mL tubes and stored at ˜80 prior to extraction. When possible at least 8×10⁷ cells were used for 4 separate genomic DNA isolation which were pooled to account for any variation with pellet size. DNA isolation was performed using the Puregene cell and tissue kit (Qiagen) with the addition of RNase A Solution (Qiagen) according to manufacturer's instructions. DNA concentration was measured in quadruplicate using either a nanodrop spectrophotometer (Thermo), Qubit dsDNA assay (Life Technologies) and the average DNA content per cell was determined.

Library preparation. The minimum number of cell equivalents of gDNA to maintain equal coverage was used for library preparation. In all screens, the minimum coverage based on cell number was multiplied by the average gDNA content per cell for each individual cell line to determine the minimum number for 10 μg PCR reactions needed to maintain coverage. A minimum coverage of 500-fold per sgRNA in the library was targeted for each independent sample or replicate but this was increased in some cases where screening was carried out with greater depth.

Library preparation was performed using a pool of eight P5 primers with to introduce a stagger in reads associated with each library and sample specific P7 primer that contained a unique sample index sequence for each timepoint, replicate, or treatment condition to be sequenced in the same pool. All library preparation primers were resuspended at 100 μM. Each library preparation PCR reaction contained the following components: 1 μl Herculase II Fusion Enzyme (Agilent), 2.5 μl Deoxynucleotide (dNTP) Solution Mix (New England Biolabs), 0.5 μl P5 primer pool, 0.5 μl P7 index primer, 20 μl 5× Reaction Buffer (Agilent), 10 μg of gDNA and nuclease-free water to bring the total reaction volume to 100 μl. Samples underwent 23 cycles of thermal cycling followed by a quality assessment by electrophoresis on 2% agarose gel to ensure consistent library amplification across multiple wells and samples for each plate. Each unique library had 10 μl pooled from all PCR reactions performed on that unique sample and mixed thoroughly. 50-100 μl of the pooled library preparation reactions was used to perform magnetic bead-based purification and elimination of any residual free primer using a 0.8× ratio SPRIselect beads (Beckman Coulter) according to the manufacturer's instructions. Libraries were then assessed for appropriate amplicon size and complete elimination of free primer peaks using the High Sensitivity ScreenTape Assay on the Tape Station2200 (Agilent) and quantified using the qPCR-based quantification in order to ensure only NGS-compatible amplicon was quantified using the Library Quant ROX Low Kit (Kapa Biosystems) on a QuantStudio 6 Realtime PCR System (ThermoFisher). Following qPCR quantification, all libraries were normalized to a standard concentration (typically 20-40 nM) depending on the lowest concentration library to be pooled, and then requantified by qPCR to ensure all samples were within ˜10-20% of the pool mean target concentration. After confirming accurate library quantification and normalization, samples were pooled at an equimolar ratio and submitted for sequencing. Libraries were sequenced on the NovaSeq 6000 instrument (Illumina) (150 bp, paired-end).

CRISPR screening bioinformatic pipeline and analysis. sgRNA counts were extracted directly from R1 raw sequence reads using a custom perl script that uses regular expression string matching to exactly match sgRNA sequence flanked by 10 bases of vector sequence. The vector sequence was allowed to have one error before and after the sgRNA sequence. sgRNAs were tabulated for each sample based on the sgRNA sequence. The sgRNA IDs of the Brunello library were updated to current HGNC gene names using the Total Approved Symbols. Transcript IDs were matched when possible and when matches were not found, past symbols and aliases were updated to current names. Finally, 5 sgRNAs with missing updated gene names were manually curated using literature searches. Library distribution was calculated using the caRpools R package. The DESeq2 R package was used to calculate differential abundance of genes. Gene counts were generated using the sum of counts for sgRNAs of the same gene. Synthetic lethality compared GemCis day 19 and GemCis day 25 vs. PBS day 19 and PBS day 25 with the day as a covariate. In the comparison integrating all cell lines, cell line was additionally modeled as a covariate. Gene essentiality was calculated by comparing PBS day 25 to PBS day 0 and in the integrated all cell lines comparison; cell line was modeled as a covariate. Common synthetic lethal genes were defined as being statistically significantly differentially lost (FDR<0.05 and Log 2 FC<0) in each of the 5 cell lines. Gene set enrichment analysis (GSEA) was performed using the fgsea R package run with 10000 permutations with the KEGG and Reactome gene sets from MSigDB. Heatmaps were generated with the ComplexHeatmap R package following z-score transformation. Other plots were generated using the ggplot2 R package.

Xenograft experiments. Six-week-old, female NU/J mice (Jackson Labs) were allowed to acclimate for at least one week prior to initiating any experiments. Mice had free access to food and water in pathogen-free housing. For KU1919-GC xenografts, cells that had been stably transduced with non-targeting control (shCtrl1, SHC002) and NPEPPS (shN39, TRCN0000073839) shRNA constructs. Mice were divided into groups of 22 and 23 for the non-targeting control and NPEPPS shRNA constructs respectively. Mice were injected with 4×10⁶ cells in phenol red- and serum-free RPMI mixed with equal volume Matrigel Matrix (Corning) to total 100 μl volume. Tumors were allowed to engraft for 9 days following injection and mice were randomized based on tumor volume within each shRNA condition into groups of 11 or 12 to be treated with combination gemcitabine plus cisplatin or DPBS. Treatment was initiated 13 days post-inoculation with dosing adjusted based on individual mouse weight.

Cisplatin (Sigma) and gemcitabine hydrochloride (BOC Sciences) were both resuspended in 0.9% saline and stored protected from light at −80° C. as individual aliquots. Prior to treatment fresh aliquots of gemcitabine and cisplatin were thawed and diluted to their final concentration with 1×DPBS (Gibco). Mice were treated three times weekly on a Monday, Wednesday and Friday schedule for four weeks total. All mice in the gemcitabine plus cisplatin treated groups were given 50 mg/kg gemcitabine and 2 mg/kg cisplatin that were mixed and administered as a single intraperitoneal injection, while control mice were administered an equivalent volume of DPBS.

Mouse health was monitored daily and all tumor volume measurements and weights were measured 3× weekly schedule. Tumor volume was calculated using the formula (L×W²)/2, for which L is the length of the long axis and W is the width of the axis perpendicular to the long axis measurement. All measurements were performed using digital calipers. Animal were humanely euthanized with CO₂ followed by cervical dislocation when tumors reached a predetermined endpoint of 2 cm³ or when weight loss exceeded 15% body weight. Mice that were removed from study due to weight loss were censored in the survival analyses.

Linear mixed-effects model of tumor growth. Linear mixed-effects models were used to model longitudinal observations of xenograft tumor growth volumes normalized by their corresponding baseline volume. Mixed-effects models from the R-package lme4 and Satterthwaite's approximation for degrees of freedom for the fixed effects from lmerTest were used for model fitting and inspection in the R statistical software (4.0.3). Volume changes compared to baseline were log 2-transformed. The final model was structured as:

$${log}_{2} ( \frac{{y}_{i , t}}{{γ}_{i , :}} ) {β}_{0} + {β}_{1} {x}_{i , t} + {β}_{2} {x}_{i , t}^{2} {β}_{3} {x}_{i , t} {KD}_{i} + {β}_{4} {x}_{i , t} {GC}_{i} + {β}_{5 i} {x}_{i , t} {KD}_{i} {GC}_{i} + {γ}_{0 , i} + {γ}_{1 , i} {x}_{i , t} + {ε}_{i , t}$$

where β is the fixed effects capturing population-level trends, γ is the normally distributed random effects capturing individual-level variation, ε is the i.i.d. normally distributed residual term, i is the unique individual identifier, t notes the time points, x_{i,t}∈{2, 4, 5, 7, 9, 11, 14, 16, 18, 21, 23, 25, 28} depicted days since initiating interventions, y_{i,:} is tumor volume at baseline prior to treatments upon randomization, and y_{i,t} were the observed tumor volumes over the treatment period measured in mm³. The model was fit using Restricted Maximum Likelihood and built iteratively until the underlying model assumptions and model convergence criteria were met. To this end, a quadratic growth term (β₂) was added on top of the linear growth term (β₁) and intercept (β₀), allowing slightly non-linear relative growth patterns to be captured by the otherwise linear model. Binary indicators KDᵢ∈{0,1} and GCᵢ∈{0,1} were used to model knockdown of NPEPPS, GemCis treatment, or the combination. The corresponding model terms were captured in β₃, β₄ and β₅, respectively. Finally, the model allows for individual-specific random effects for intercept (γ_{0,i}) and linear growth slope (γ_{1,i}). Shapiro-Wilk test was used to examine the underlying normality assumption for γ_{0,i} and γ_{1,i} with p=0.1373 and p=8901, respectively, indicating that these random effects followed underlying assumptions of normality. After inspection of the residual plots (FIGS. 11E-11F), this final model was deemed suitable for population-level statistical inference via the fixed effects. This population-level model fits are visualized in FIGS. 11A-11D. These population-level estimates are as provided in Table 6.

TABLE 6

| Fixed effect | Estimate | Std. error | df | t | p-val |
|---|---|---|---|---|---|
| β₀ (intercept) | 0.05054 | 0.08422 | 54.28 | 0.600 | 0.55091 |
| β₁ (linear slope) | 0.1236 | 0.01493 | 65.52 | 8.276 | 8.92e−12 *** |
| β₂ (quadratic slope) | 0.00308 | 0.0002242 | 389 | 13.740 | <2e−16 *** |
| β₃ (knockdown) | −0.0605 | 0.01821 | 44.97 | −3.322 | 0.00178 ** |
| β₄ (GC) | −0.1063 | 0.01821 | 44.97 | −5.837 | 5.49e−07 *** |
| β₅ (knockdown + GC) | −0.1233 | 0.01791 | 45.28 | −6.884 | 1.47e−08 *** |

Survival analyses from TCGA. Copy number and gene expression data for patients with muscle-invasive bladder cancer in the TCGA cohort (PanCancer Atlas) were downloaded from cBioPortal. Patients were separated into treatment groups, platinum-based treatment (n=87) or unrecorded treatment (n=204), and then stratified based on copy number gain or amplification, or mRNA upregulation (z-score >1) of LRRC8A or LRRC8D. The Logrank test was used to test the difference in overall survival between the stratified patient groups.

Tumor-derived Organoids. Culture of the organoids. Human bladder tissue was obtained from the Erasmus MC Bladder Cancer Center, Rotterdam, the Netherlands. Bladder tumor-derived organoids from biopsies obtained through TURBT or cystectomy. Briefly, bladder tissues were washed with Advanced DMEM/F12 (Gibco) supplemented with 10 mM HEPES (Gibco), 1% GlutaMax (Gibco) and 100 μg/ml primocin (InvivoGen), henceforth Ad+++. Tissue was minced and incubated at 37° C. with the digestion solution (collagenase 2.5 mg/ml in EBSS) and isolated cells were passed through 70 μM strainer (Falcon), washed with Ad+++ and seeded in 50 μl drops of BME (R&D system) containing 10000-15000 cells in 24 well suspension plates (Greiner). Bladder tumor organoids were cultured in a culture medium containing Ad+++ supplemented with 1×B-27 (Gibco), 1.25 mM N-acetylcysteine (Sigma), 10 mM nicotinamide, 20 μM TGFβ receptor inhibitor A83-01, 100 ng/ml recombinant human FGF10 (Peprotech), 25 ng/ml recombinant human FGF7 (Peprotech), 12.5 ng/ml recombinant human FGF2 (Peprotech), 10 μM Y27632 Rho Kinase (ROCK) Inhibitor (Sigma) and conditioned media for recombinant Rspondin (2.5% v/v), and Wnt3A (2.5% v/v). The medium was changed every three days. Organoids were passaged at a 1:3 to 1:6 ratio every 7 days using cell dissociation solution-non enzymatic (Sigma) and plated in fresh BME matrix droplets.

Drug screening. Organoids were collected 7 days after passaging, passed through a 100 μM strainer and 1000 organoids were seeded per well of a 48-well plate in BME matrix droplets. After 24 hours cisplatin (Sigma) resuspended in PBS was added at different concentrations (2, 10, 25, and 50 μM) with or without tosedostat (20 μM) (Tocris) resuspended in DMSO. All wells were adjusted to contain less than 0.7% DMSO. Organoids were cultured for the first 6 days in the presence of drugs followed by drug withdrawal, where organoids were grown in organoid culture media for 10 days. The entire content of the wells in different treatment groups was collected, washed and reseeded after disaggregation in fresh BME, and cultured for 6 days. Cell viability was assayed using alamarBlue (Invitrogen) according to the manufacturer's instructions after 6 days of drug incubation, 10 days of drug withdrawal, and 6 days post reseeding. Viability data was normalized using organoid wells treated with vehicle control.

SNaPshot mutation and microarray analysis. Tumor, organoid, and matched normal DNA were isolated using with the QIAmp DNA Mini-Kit (Qiagen) according to the manufacturer's protocol. Presence of hotspot mutations in the TERT promoter sequence chr5:1,295,228C>T, chr5:1,295,248 G>A and chr5:1,295,250C>T [GRCh37/hg19]), FGFR3 (R248Q/E, S249C, G372C, Y375C, A393E, K652E/M) and PIK3CA (E542K, E545 G/K and H1047R) were assessed on tumor, normal and organoid DNA by SNaPshot mutation analysis. Copy number aberration analysis was performed using single-nucleotide polymorphism (SNP) microarrays (Infinium Global Screening Array (GSA) V3, Illumina) on primary tumor DNA, matched DNA collected from non-tumor urothelium plus stromal tissue from the same sample but from a distant location from the tumor, and organoid DNA using standard protocols. SNP data (log-R ratio, B-allele frequency) were visualized to identify potential CNVs via Biodiscovery Nexus CN7.5. (Biodiscovery) and the GenomeStudio genotyping module (Illumina).

Organoid phenotyping and tumor histology. Tissue processing and H&E staining was performed using standard procedures. For hematoxylin-eosin (H&E) staining of organoids, wells of BME-embedded organoids were fixated with 4% formalin (Sigma) and 0.15% glutaraldehyde (produced in-house) at room temperature for 2 hours. Fixated BME and organoids were washed with PBS and engulfed in 2.5% Low-Melting Agarose (Sigma) prior to paraffin embedding. H&E staining was performed on 4 μM paraffin sections of both tumor and organoid tissue. Stained whole-slides, as well as prior 3D organoid cultures were imaged by bright-field microscopy (Olympus IX70).

All of the COMPOSITIONS and METHODS disclosed and claimed herein can be made and executed without undue experimentation in light of the present disclosure. While the COMPOSITIONS and METHODS have been described in terms of preferred embodiments, it will be apparent to those of skill in the art that variation can be applied to the COMPOSITIONS and METHODS and in the steps or in the sequence of steps of the METHODS described herein without departing from the concept, spirit and scope of the invention. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating a tumor, comprising administering to a subject in need thereof an M1 aminopeptidase inhibitor; wherein the subject is undergoing or will undergo an anti-cancer therapy comprising one or more platinum-based chemotherapeutic agent and sensitizing the tumor to platinum-based chemotherapeutic agents in the subject.

2. The method according to claim 1, wherein the tumor comprises a platinum-resistant metastatic solid tumor.

3. The method according to claim 1, wherein the tumor comprises a testicular tumor, ovarian tumor, cervical tumor, kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma or a combination thereof.

4. The method according to claim 1, wherein the one or more platinum-based chemotherapeutic agent comprises one or more of cisplatin, carboplatin, nedaplatin, satraplatin, picoplatin, phenanthriplatin and triplatin tetranitrate.

5. The method according to claim 1, wherein the M1 aminopeptidase inhibitor comprises a puromycin-sensitive aminopeptidase (NPEPPS) inhibitor.

6. The method according to claim 1, wherein the M1 aminopeptidase inhibitor comprises tosedostat.

7. The method according to claim 1, wherein the tumor comprises a bladder tumor.

8. (canceled)

9. A method for treating a solid tumor, comprising administering to a subject in need thereof an effective amount of an M1 aminopeptidase inhibitor when the subject is undergoing an anti-cancer therapy comprising one or more platinum-based chemotherapeutic agent, wherein puromycin-sensitive aminopeptidase (NPEPPS) gene expression or protein level is measured in a tumor biopsy sample from the subject at least one of before, during, and after the treatment.

10. The method according to claim 9 wherein the solid tumor is a metastatic solid tumor.

11. The method according to claim 9, wherein solid tumor comprises testicular tumor, ovarian tumor, cervical tumor, kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma or a combination thereof.

12. The method according to claim 9, wherein the one or more platinum-based chemotherapeutic agent comprises one or more of cisplatin, carboplatin, nedaplatin, satraplatin, picoplatin, phenanthriplatin, triplatin tetranitrate, or a combination thereof.

13. The method according to claim 9, wherein the aminopeptidase inhibitor comprises a puromycin-sensitive aminopeptidase (NPEPPS) inhibitor.

14. The method according to claim 9, wherein the M1 aminopeptidase inhibitor comprises tosedostat.

15-19. (canceled)

20. The method according to claim 18, further comprising adjusting the dosing regimen by increasing or decreasing the dosing regimen or dosing concentration of the M1 aminopeptidase inhibitor, the dose of the one or more platinum-based chemotherapeutic agent, or both, when an adverse effect is observed.

21. A composition for treating cancer comprising: a composition comprising one or more M1 aminopeptidase inhibitors and one or more platinum-based chemotherapeutic agent.

22. The composition according to claim 21, wherein the one or more M1 aminopeptidase inhibitors comprises a puromycin-sensitive aminopeptidase (NPEPPS) inhibitor.

23. The composition according to claim 21 , wherein the one or more platinum-based chemotherapeutic agent comprises one or more of cisplatin, carboplatin, nedaplatin, satraplatin, picoplatin, phenanthriplatin, triplatin tetranitrate, or a combination thereof.

24. The composition according to claim 21, wherein the one or more M1 aminopeptidase inhibitor comprises tosedostat and the one or more platinum-based chemotherapeutic agent comprises carboplatin.

25. The composition according to claims 21, further comprising one or more of gemcitabine, methotrexate, vinblastine, and adriamycin.

26. (canceled)

27. A kit comprising at least one M1 aminopeptidase inhibitor, at least one platinum-based chemotherapeutic agent, and at least one container.
